(19) United States
(12) Patent Application Publication
(10) Pub. No.: US 2026/0248829 A1
(43) Pub. Date: Aug. 27, 2026

(54) PHOSPHOLIPID COMPOSITIONS COMPRISING EPA AND/OR DHA FOR AMELIORATING NEURODEGENERATION AND AGE-RELATED NEURODEGENERATIVE DISORDERS

(71) Applicant: Aker BioMarine Human Ingredients AS, Lysaker (NO)

(72) Inventor: Line JOHNSEN, Lysaker (NO)

(21) Appl. No.: 18/879,087

(22) PCT Filed: Jun. 26, 2023
(86) PCT No.: PCT/IB2023/056584

§ 371 Date: Dec. 26, 2024

(30) Foreign Application Priority Data
Jun. 27, 2022 (GB) 2209414.8

(51) Int. Cl. A61K 31/683; A61K 9/48; A61P 25/28

(52) U.S. Cl. CPC A61K 31/683; A61K 9/4825; A61P 25/28

(57) ABSTRACT

The present invention relates to compositions comprising marine derived phospholipids for ameliorating at least one aging hallmark selected from the group consisting of genomic instability, telomere shortening, an epigenetic alteration, loss of proteostasis, deregulated nutrient sensing, mitochondrial dysfunction, cellular senescence, stem cell exhaustion, and deregulated nutrient sensing and intercellular communication.

### FIELD OF THE INVENTION

The present invention relates to compositions comprising marine derived phospholipids for ameliorating at least one aging hallmark selected from the group consisting of genomic instability, telomere shortening, an epigenetic alteration, loss of proteostasis, deregulated nutrient sensing, mitochondrial dysfunction, cellular senescence, stem cell exhaustion, and deregulated nutrient sensing and intercellular communication.

### BACKGROUND OF THE INVENTION

The UN estimates that 1 in 6 people (globally) will be over the age of 65 (16%) by 2050, up from 1 in 11 (9%) in 2019. The improvement in life expectancy has, however, not been accompanied by the same increase in health span. Since aging is one of the main risk factors for most chronic pathologies, the prevalence of age-related disease has risen with the increasing average lifespan, representing a socio-economic problem in developed societies. It is believed that multi-morbidity prevalence in incoming cohorts aged 65-74 years will rise from 45.7% in 2015 to 52.8% in 2035 (Andrew Kingston, 2018). Therefore, unless combined with an enhanced health span, increased longevity can translate into more years of misery and suffering, as we spend more years unable to autonomously perform the normal activities of daily living. A recent, piloted objective population-wide NHS study of data reflected that currently women may live for 29 years in poor health and men for 23 years (Garth, 2020) in a developed western society such as the UK.

Age also tends to come with a greater desire to live healthily for as long as possible. It is thus expected that as the population of older generations grow, they will likely direct their expenditure toward supplements that protect cognition, maintain vitality and slow physical decline, creating a need for longevity supplements with proven effects.

Longevity supplements differ from other “generic supplements” as they do not just provide ingredients to the body simply to prevent deficiencies (such as for example vitamin or mineral deficiencies); longevity supplements are able to provide the body with ingredients that can act on pathways to change the rate at which we age.

Aging is a biological process characterized by progressive loss of viability and increasing frailty. For humans, aging can be considered as a continuous decline of fitness and an intrinsic loss-of-function. One specific effect of normal aging in humans is the loss of neurons, known as normal age-related neurodegeneration. As aging also is the primary risk factor for many of the major age-related diseases, particularly for most of the neurodegenerative diseases (NDDs), it is generally accepted that slowing aging may reduce the incidence of age-related diseases. In fact, there is mounting evidence that interfering with the conserved processes that induce aging can extend healthy lifespan.

The steep rise in these NDDs worldwide is about to impose a massive health care challenge. Currently drugs to treat such NDDs are limited to cure symptoms for a short period of time. There is a pressing and unmet need to find a novel drugs and supplements that can slow the course of aging and the accompanied disease progression. It is believed that attenuating the aging process may delay the onset and slow progression of the major NDDs.

Many cellular processes contribute to aging and these are referred to as “hallmarks of aging”.

It would be beneficial if anti-aging therapies could demonstrate effects on one or more of the hallmarks of aging.

### SUMMARY OF THE INVENTION

Accordingly, in some preferred embodiments, the present invention relates to a marine derived phospholipid composition for use in therapy (prophylactic or therapeutic) and a method for ameliorating one or more symptoms and/or signs of aging.

In some preferred embodiments, the present invention provides methods for ameliorating at least one aging hallmark in a subject comprising administering to a subject in need thereof an effective amount of a composition comprising phospholipids having bound eicosapentaenoic acid (EPA) and/or docosahexaenoic acid (DHA), wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication.

In some preferred embodiments, the present invention provides methods for improving dopaminergic neuron survival in a subject in need thereof comprising administering to the subject in need thereof an effective amount of a composition comprising phospholipids having bound eicosapentaenoic acid (EPA) and/or docosahexaenoic acid (DHA). In some preferred embodiments, the administration of the effective amount of the composition comprising phospholipids having bound eicosapentaenoic acid (EPA) and/or docosahexaenoic acid (DHA) improves clearance of α-synuclein aggregation in the subject.

In some preferred embodiments, the present invention provides a composition comprising phospholipids having bound EPA and/or DHA for use in ameliorating an aging hallmark selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication. In some preferred embodiments, the invention provides a composition comprising phospholipids having bound EPA and/or DHA for use in treating (prophylactic or therapeutic) a disease or condition associated with an aging hallmark. In another embodiment, administration of the composition is sufficient to ameliorate at least one of the aging hallmarks or for use in improving dopaminergic neuron survival.

The invention also provides the use of a composition comprising phospholipids having bound EPA and/or DHA in the manufacture of: (i) a medicament for ameliorating an aging hallmark selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication or for improving dopaminergic neuron survival; or (ii) a medicament for treating (prophylactic or therapeutic) a disease or condition associated with an aging hallmark or degeneration of dopaminergic neurons. Administration of the medicament may be sufficient to ameliorate at least one of the aging hallmarks.

In some preferred embodiments, the composition comprising phospholipids having bound EPA and/or DHA is characterized in comprising from 5 to 80 g/100 g, from 5 to 60 g/100 g, from 5 to 40 g/100 g, or from 10 to 40 g/100 g EPA and/or DHA as assayed by gas chromatography fatty acid methylester (GC-FAME) analysis. In some preferred embodiments, the composition comprising phospholipids having bound EPA and/or DHA is characterized in comprising from 10 to 70%, 10 to 50%, or 10 to 40% EPA and/or DHA w/w where w/w is determined by measuring the total weight of EPA and/or DHA residues in the composition and dividing the total weight of EPA and/or DHA residues by the total weight of the composition.

In some preferred embodiments, the composition comprising phospholipids having bound EPA and/or DHA is characterized in having a ratio of EPA to DHA of from 1:1 to 5:1 and preferably from 1:1 to 3:1. In some preferred embodiments, the composition comprising phospholipids having bound EPA and/or DHA is characterized in having a ratio of DHA to EPA of from 1:1 to 5:1 and preferably from 1:1 to 3:1.

In some preferred embodiments, the composition comprising phospholipids having bound EPA and/or DHA is further characterized in having a total phospholipid content of from 10 to 90% w/w, 10 to 80% w/w, 20 to 80% w/w, or 20 to 60% w/w, where w/w is determined by measuring the total weight of phospholipids in the composition by 31P-NMR and dividing the total weight of the phospholipid by the total weight of the composition.

In some preferred embodiments, the composition comprising phospholipids having bound EPA and/or DHA is further characterized in having a choline content of from 2 to 20% w/w, 2 to 15% w/w, or 2 to 10% w/w, where w/w is determined by measuring the total weight of choline in the composition by 31P-NMR and dividing the total weight of the phospholipid by the total weight of the composition.

In some preferred embodiments, the composition comprising phospholipids having bound EPA and/or DHA is further characterized in comprising astaxanthin esters in an amount of from 50 to 1000 mg/kg, 50 to 500 mg/kg, or 50 to 300 mg/kg, or 100 to 300 mg/kg.

In some preferred embodiments, the composition comprising phospholipids having bound EPA and/or DHA is further characterized in having a TMAO content, expressed as mgN/100 g, of from 1 to 30, 1 to 20, or 1 to 10 mgN/100 g.

In some preferred embodiments, the composition comprising phospholipids having bound EPA and/or DHA is further characterized in a peroxide value of less than 10, 5, or 2.

In some preferred embodiments, the composition comprising phospholipids having bound EPA and/or DHA is further characterized in having an acid value of less than 50, 40, 30 or 20.

In some preferred embodiments, the composition comprising phospholipids having bound EPA and/or DHA is further characterized in having a Na+ content of less than 1.000%, 0.500%, 0.300% or 0.200% w/w of the composition, where w/w is determined by measuring the total weight of Na+ in the composition and dividing the total weight of Na+ by the total weight of the composition.

In some preferred embodiments, the composition comprising phospholipids having bound EPA and/or DHA is further characterized in having a phosphatidylcholine (PC) content of from 20 to 70%, 20 to 60%, or 20 to 50% w/w, where w/w is determined by measuring the total weight of PC molecules with two fatty acids attached in the composition and dividing the total weight of PC molecules with two fatty acids attached by the total weight of the composition.

In some preferred embodiments, the composition comprising phospholipids having bound EPA and/or DHA is further characterized in having a lysophosphatidylcholine (LPC) content of from 10 to 70%, 10 to 60%, or 10 to 50% w/w, where w/w is determined by measuring the total weight of LPC molecules in the composition with one fatty acid attached and dividing the total weight of LPC molecules with one fatty acid attached by the total weight of the composition.

In some preferred embodiments, the composition comprising phospholipids having bound EPA and/or DHA is prepared from a natural source. In some preferred embodiments, the natural source is selected from the group consisting of krill, fish or fish by-products, Calanus, shrimp, mollusks, cephalopods, and algae. In some preferred embodiments, the natural source is krill. In some preferred embodiments, the fish or fish by-products are derived from herring, salmon, anchovy or mackerel.

In some preferred embodiments, the composition comprising phospholipids having bound EPA and/or DHA is an oil.

In some preferred embodiments, the composition comprising phospholipids having bound EPA and/or DHA is encapsulated.

In some preferred embodiments, the composition comprising phospholipids having bound EPA and/or DHA is encapsulated in a gel capsule.

In some preferred embodiments, the composition comprising phospholipids having bound EPA and/or DHA comprises an antioxidant and/or flavoring agent that does not naturally occur in the source of the phospholipid composition.

In some preferred embodiments, the composition comprising phospholipids having bound EPA and/or DHA is provided as a medical food.

In some preferred embodiments, the compositions are provided to a subject in need thereof. In some preferred embodiments, the subject in need thereof has or is at risk of a disease or condition selected from the group consisting of age-related neurodegeneration or disorders, Alzheimer's disease, Parkinson's disease, decline in executive function, age-related cognitive decline, a cognitive disease, disorder or impairment, and senescence related to age-related pathologies selected from the group of atherosclerosis, cardiovascular dysfunction (including cardiovascular problems caused by certain genotoxic chemotherapies), tumor progression, loss of hematopoietic and skeletal muscle stem cell functions, non-alcoholic fatty liver disease, pulmonary fibrosis, osteoarthritis and osteoporosis.

### BRIEF DESCRIPTION OF THE DRAWINGS

FIG. 1a-1f. Krill oil promotes healthy dopaminergic neuronal aging. (a) Representative images of the head region of PD animals at day 1, day 3 and day 6 of adulthood, Scale bar, 20 μm. (b) Survival of anterior CEPs and ADEs DA neurons of PD nematodes co-expressing human α-synuclein ((α-syn) during aging in response to Krill oil (n=35 nematodes per experiment; three independent experiments, s.e.m; ***p<0.001; one-way ANOVA followed by Bonferroni's multiple comparison test. (c) Representative aggregation of α-SYN in the body wall muscles in day 1 and day 6 animals, Scale bar, 20 μm. (d) The quantification of number of aggregates in day 1 and day 6 animals in response to krill oil (n=15 nematodes, s.e.m; NS and ***p<0.001; one-way ANOVA followed by Bonferroni's multiple comparison test). (e) The column scatter plot represents basal slowing response of wild type animals and PD at adult day 6. Body bends per 20 second measured on NGM plates with and without bacteria (n=30; Error bars, s.e.m; ***p<0.001; one-way ANOVA followed by Bonferroni's multiple comparison test. (B) The column scatter plot represents locomotion activity wildtype and PD animals at their adult day 6. The activity was scored for 60 minutes (n=50 animals per experiment; Error bars, s.e.m; NS and *p<0.05; one-way ANOVA followed by Bonferroni's multiple comparison test.

FIG. 2a-2e. Krill oil delays senescence via p21 and TGFβ. (α-b) Representative images of the β-gal staining of the head region of wildtype and PD animals at their adulthood of 9 days treated with and without Krill oil. The column scatter plot represents percentage of worms with positive senescence mark in three independent experiments (n=50-100 individuals, column indicates mean, error bars, s.e.m, ***p<0.001; one-way ANOVA followed by Bonferroni's multiple comparison test). (c-d) Image and quantification represents senescence in BJ cell line using β-gal staining in response to krill oil (three independent experiments, Error bars, s.e.m; *p<0.05; one-way ANOVA followed by Bonferroni's multiple comparison test). (e) Relative p21 and TGFβmRNA levels in BJ cells at passage 10 and 21 treated with Krill oil (100 μg/ml, 6 days) as measured by qPCR. Data represent means±s.d., n=3. *P≤0.05, **P≤0.01, ****P≤0.0001 (two-tailed Student's t-test).

FIG. 3a-3e. Krill oil improves mitochondrial health. (α-b) Represents the image and quantification of 8-Oxo (dG) staining in old day 6 PD animals in response to krill oil (n=10 individuals, Error bars, s.e.m; ****p<0.0001; one-way ANOVA followed by Bonferroni's multiple comparison test).(c) Oxygen consumption rate in day 1 and day 6 Wildtype and PD animals in response to krill oil (n=50 individuals, two independent experiments, Error bars, s.e.m; NS and *p<0.05,**p<0.01; one-way ANOVA followed by Bonferroni's multiple comparison test). (d-e) image and quantification of mitochondrial membrane potential measured using TMRE staining in BJ fibroblast passage 16 (p16) in absence and presence of krill oil (n=6 independent experiments, Error bars, s.e.m; **p<0.01; one-way ANOVA followed by Bonferroni's multiple comparison test).

FIG. 4a-4c. krill oil alters genes regulation. (a) In this PCA plot all the time points are depicted in the same plot for BY273 strain only in control and krill oil treated animals. The condition variation between day 1 animals are quite low, whereas in day 3 and day 6 animal's variance is almost 22%. Clearly with aging krill oil alters genes regulation. (b) Represents differentially expressed genes (DEG) in PD animals in presence and absence of krill oil treatment in day 1, day 3 and day 6 animal. (c) Represents the volcano plot of significant genes upregulated and downregulated in PD animals treated with krill oil in three different time point day1, day3 and day 6.

FIG. 5a-5c. Krill oil stimulates gene clusters. (a) Represents upregulated clusters 6, 7, 8 and 12, in PD animals treated with krill oil in three different time points day 1, day3 and day6. (b) The GOs, KEGG, HP of cluster 8 cluster. (c) The GOs, KEGG, HP of cluster 12 cluster.

FIG. 6a-6b. Krill oil promoted DA neuron aging via cnnm-3, pbo-2 and rim-1 in PD animals. (α-b) Image and scatter dot plot represents intensity of the dopaminergic neurons after depleting cnnm-3, pbo-2 and rim-1 in PD animals (n=14 individuals, Scale bar, 20 μm Error bars, s.e.m; ***p<0.001; one-way ANOVA followed by Bonferroni's multiple comparison test).

FIG. 7a-7c. WGCNA analysis links positive and negative correlation between gene expression and Krill oil dependent parameters. (a) the heatmap shows you the correlation values between all of the found modules (gene groups) and defined parameters (krill oil treated, DA neuron survival, OCR and locomotion). The correlation range from +1 (completely positively correlated) to −1 (perfect negative correlation).(b-c) Represents gprofiler plots to investigate each module (b) The GOs, KEGG, HP of green module. (c) The GOs, KEGG, HP of blue module.

FIG. 8a-8c. Krill oil upregulates CNNM3, PLCB4 and RIMS1 in human BJ cell line. (a-c) Relative mRNA levels of differentially expressed genes (DEGs) in BJ cells treated with Krill oil (100 μg/ml) for different time (1, 3 and 6 days) as measured by qPCR. Data represent means±s.d., n=3. *P≤0.05, **P≤0.01, **P≤0.001, ****P≤0.0001 (two-tailed Student's t-test).

FIG. 9a-9d. Krill oil protects dopaminergic neurons via oxidative stress regulators. (a) Representative images of the head region of PD animals at day 6 of adulthood in control and krill oil treated condition following knockdown of jnk-1, skn-1, hmg-5 and lmd-3 Scale bar, 20 μm. (b) The scatter dot plot represents GFP intensity of the CEPs dopaminergic neurons in old day 6 PD nematodes following knockdown of jnk-1, skn-1, hmg-5 and lmd-3 (n=20 individuals; Error bars, s.e.m; ***p<0.001; one-way ANOVA followed by Bonferroni's multiple comparison test). (c) Quantification of pharyngeal pumping frequency (Hz) monitored in wildtype and PDa animals using the nemamatrix screenchip based assay. The column scatter plot represent the pharyngeal pumping frequency of adult day 3 animals (n=15-20 individuals, Error bars, s.e.m NS and **P≤0.001, ****p<0.0001; one-way ANOVA followed by Bonferroni's multiple comparison test). (d) The learning index was calculated from a positive association with butanone in wildtype and PD animals with and without Krill oil at adult day 6 (n=150 individuals, two independent experiments, error bars, s.e.m; NS, * p<0.05; one-way ANOVA followed by Bonferroni's multiple comparison test)

FIG. 10. Krill oil (such as Superba Krill Oil) improves aging by promoting genomic stability, mitochondrial health and suppresses senescence and proteostatis loss. This graphical representation demonstrates the benefit of krill oil and the pathways involved which leads to protection of DA neurons using C. elegans and human BJ fibroblast cells as a model.

### DEFINITIONS

Throughout the present disclosure relevant terms are to be understood consistently with their typical meanings established in the relevant art, i.e., the art of pharmaceutical chemistry, medicine, biology, biochemistry and physiology.

The term “EPA” refers to eicosapentaenoic acid.

The term “DHA” refers to docosahexaenoic acid.

EPA and DHA, as used herein in connection with the compositions of the invention, refers to the fatty acid chain that can be bound to a lipid backbone, such as to phospholipids, lysophospholipids, triacylglycerides, diacylglyceride, monoacylglyceride or any other lipid backbone, or it can exist in the compositions as a free fatty acid or ethyl ester.

The term “hallmarks of aging” (used synonymously with “age-related hallmark” and “hallmark related to aging”) is an art-recognized term. In certain embodiments, the term refers to the cellular processes that contributes to aging, including, without limitation, genomic instability, epigenetic alteration, mitochondrial dysfunction, loss of proteostasis, senescence, telomere shortening, deregulated nutrient sensing (altered metabolism) and cell-cell communication, as well as stem cell exhaustion. These hallmarks are known, and reviewed in depth by C. Lopez Otin, et al.2013. Excerpts of Lopez Otin, 2013, is given below.

As used herein, the term “ameliorate”, when used in reference to an aging hallmark, includes, without limitation, (i) slowing, stopping, reversing, or preventing the hallmark's progression, (ii) slowing, stopping, reversing, or preventing the progression of the hallmark's symptoms, (iii) preventing or reducing the likelihood of the hallmark's recurrence, and/or (iv) preventing or reducing the likelihood that the hallmark's symptoms will recur. In one embodiment, treating a subject afflicted with an aging hallmark means (i) reversing the hallmark's progression, ideally to the point of eliminating the hallmark, and/or (ii) reversing the progression of the hallmark's symptoms, ideally to the point of eliminating the symptoms.

The term “total phospholipids” is used herein to describe the total content of phospholipids, including lyso-phospholipids, in a composition. As used herein, “phospholipid” refers to an organic compound that has two fatty acid moieties attached at the sn-1 and sn-2 positions of glycerol and contain a head group linked by a phosphate residue at the sn-3 position of the glycerol. Exemplary headgroup moieties include choline, ethanolamine, serine and inositol. Phospholipids include phosphatidylcholine, phosphatidylserine, phosphatidylethanolamine, phosphatidylinositol, and phosphatidic acid. The fatty acid moiety is the portion of the fatty acid molecule that is bound at the sn-1 or sn-2 position, for example by an ester or ether linkage. When the fatty acid moiety is a fatty acyl, the aliphatic chain of the fatty acyl is attached via an ester linkage and when the fatty acid moiety is an aliphatic chain of a fatty acid, the aliphatic chain is attached via an ether linkage. When a particular fatty acid is mentioned in connection with a phospholipid of the invention (e.g., EPA or DHA) it should therefore be taken as a reference to the relevant fatty acyl group or to its aliphatic chain. In krill oil, a predominant amount of the total amount of EPA and/or DHA is bound to a phospholipid, in particular a predominant amount of EPA and/or DHA is bound to phosphatidylcholine.

The term “total phosphatidylcholine” is used herein to describe the total content of phosphatidylcholine, including lyso-phosphatidylcholine (LPC), in a composition.

The term “total LPC” is used herein to describe the total content of lyso-phosphatidylcholine in a composition.

The term “pharmaceutically acceptable excipients” refer to substances different from the components of the LPC-compositions referred to in the claims and which are commonly used with oily pharmaceuticals. Such excipients include, but are not limited to triolein, soybean oil, safflower oil, sesame oil, castor oil, coconut oil, triglycerides, tributyrin, tricaproin, tricaprylin, vitamin E, antioxidants, α-tocopherol, ascorbic acid, deferoxamine mesylate, thioglycolic acid, emulsifiers, lecithin, polysorbate 80, methylcellulose, gelatin, serum albumin, sorbitan lauraute, sorbitan oleate, sorbitan trioleate, polyethylene glycol (PEG), PEG 400, polyethylene glycol-modified phosphatidylethanolamine (PEG-PE), poloxamers, glycerin, sorbitol, Xylitol, pH adjustment agents; sodium hydroxide, antimicrobial agents EDTA, sodium benzoate, benzyl alcohol and proteins such as albumin. The pharmaceutically acceptable excipients must be acceptable in the sense of being compatible with the other ingredients of the composition and not deleterious to the recipient thereof.

Used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of pharmaceutical salts properties, Selection, and Use; 2002.

The term “prophylaxis” means measures taken to prevent, rather than treat, diseases or conditions.

The term “therapeutically effective amount” is an art-recognized term. In certain embodiments, the term refers to an amount of the composition disclosed herein that produces some desired effect at a reasonable benefit/risk ratio applicable to the medical treatment. In certain embodiments, the term refers to that amount necessary or sufficient to eliminate, reduce or alleviate medical symptoms for a period of time. The effective amount may vary depending on such factors as the disease or condition being treated, the particular composition being administered, the size of the subject, or the severity of the disease or condition. One of ordinary skill in the art may empirically determine the effective amount of a particular composition without necessitating undue experimentation.

The term “treating” is art-recognized and includes preventing a disease, disorder or condition from occurring in a subject which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition may include affecting the underlying pathophysiology of the disease or condition to bring about an overall benefit to the patient (such as inhibiting the disease, disorder or condition, e.g., impeding its progress; and relieving the disease, disorder, or condition, e.g., causing regression of the disease, disorder and/or condition). Treating the disease or condition may include ameliorating at least one or more than one symptom of the particular disease or condition, for example an overall improvement in symptoms, even if the underlying pathophysiology is not affected, such as treating dry eye disease selected from inflammation of the eye, corneal nerve abnormalities and abrasions on the surface of the eye or neurodegenerative disease of the eye selected from age-related macular degeneration, diabetic retinopathy, Non-Proliferative Retinopathy, Proliferative Retinopathy, Diabetic macular edema, Retinitis pigmentosa, Central vein occlusion and glaucoma and other related diseases or any other medical condition, is well understood in the art, and includes administration of a composition which reduces the frequency of, or delays the onset of, symptoms of a medical condition in a subject relative to a subject which does not receive the composition.

Unless specifically defined herein, all technical and scientific terms used have the same meaning as commonly understood by a skilled artisan in the fields of medicine, pharmacology, pharmaceutical chemistry, biology, biochemistry and physiology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail.

Where a numerical limit or range is stated herein, the endpoints are included. Also, all values and sub ranges within a numerical limit or range are specifically included as if explicitly written out.

Headings have been used for organizational purposes and should not be construed as limiting the subject-matter herein.

### DETAILED DESCRIPTION OF THE INVENTION

Marine oils have been extensively researched for health promoting properties. Oil from the Antarctic krill species, Euphausia Superba, are high in long-chain omega-3 fatty acids (EPA and DHA), phospholipids, choline, and astaxanthin. Both EPA/DHA and astaxanthin all have antioxidant and anti-inflammatory properties^{4,5}. In krill oil large amounts of EPA/DHA are bound to phospholipids (PL), particularly phosphatidylcholine (PC), making the fatty acids more stable and ensures efficient uptake in tissue^{6,7}. It is also demonstrated that a type of PC, lysophosphatidylcholine (LPC), is essential for transport of long-chain omega-3 fatty acids over the blood-brain-barrier (Nguyen et al, Nature, volume 509, p. 503-506 (2014) ) and thus absorption of EPA and DHA in the brain. Krill oil derived phospholipids may therefore be a nutraceutical of choice to boost health, including brain health. In support of this, short term supplementation experiments have shown that PC- and LPC-DHA/EPA have anti-inflammatory and antioxidative properties with potential to improve motor function and some cognitive properties in mice ^{8,9}. A study where mice were given a diet enriched in LPC from krill oil for 12 months, also showed improvement of memory¹⁰.

However, a study evaluating the general potential of krill derived phospholipids as a nutraceutical targeting the aging process is lacking.

### Hallmarks of Aging

Many cellular processes contribute to aging. These processes are often referred to as the “hallmarks of aging” and include genomic instability, epigenetic alteration, mitochondrial dysfunction, loss of proteostasis, senescence, telomere shortening, deregulated nutrient sensing (altered metabolism) and cell-cell communication, as well as stem cell exhaustion 1-3 .

The aging hallmarks are the type of biochemical changes that occur in all organisms that experience biological aging and lead to a progressive loss of physiological integrity, impaired function and, eventually, death. Effective anti-aging supplements should be able to attenuate at least one of the hallmarks of aging. The nine hallmarks of aging are grouped into three categories as follows:

### Primary Hallmarks (Causes of Damage):

Genome instability
Telomere shortening
Epigenetic alterations
Loss of proteostasis

### Antagonistic Hallmarks (Responses to Damage):

Deregulated nutrient sensing
Mitochondrial dysfunction
Cellular senescence

### Integrative Hallmarks (Culprits of the Phenotype):

Stem cell exhaustion
Altered intercellular communication

Primary hallmarks are the primary causes of cellular damage. Antagonistic hallmarks are antagonistic or compensatory responses to the manifestation of the primary hallmarks. Integrative hallmarks are the functional result of the previous two groups of hallmarks that lead to further operational deterioration associated with aging.

An overview based on Lopez-Otin, 2013, is provided:

### Genomic Instability

Genomic instability is caused by defects in certain processes that control the way cells divide. Accumulation of genetic damage throughout life is a common denominator of aging and increased DNA damage accumulation is also linked to premature aging diseases.

### Epigenetic Alteration

A heritable change that does not affect the DNA sequence but results in a change in gene expression. Examples include promoter methylation and histone modifications. Also called epigenetic variant and epimutation. There are multiple lines of evidence suggesting that aging is accompanied by epigenetic changes, and that epigenetic perturbations can provoke progeroid syndromes in model organisms. As an example, studies with SIRT6 demonstrate that it is an epigenetically relevant enzyme whose loss-of-function reduces longevity and whose gain-of-function extends longevity in mice.

### Mitochondrial Dysfunction

Mitochondrial dysfunction occurs when the mitochondria don't work as well as they should. This can be due to another disease, condition, or age. Most aging-related diseases, particularly, neurodegenerative diseases, have mitochondrial involvement, including Alzheimer's disease and Parkinson's disease.

### Loss of Proteostasis

The loss of proteostasis can be described as the failure of the protein building machinery of the cell and the accumulation of misfolded proteins, which is one of the root causes of age-related diseases, including Alzheimer's disease. Proteostasis involves mechanisms for the stabilization of correctly folded proteins, most prominently the heat-shock family of proteins, and mechanisms for the degradation of proteins by the proteasome or the lysosome.

### Senescence

Cellular senescence can be defined as a stable arrest of the cell cycle that can be triggered in normal cells in response to various intrinsic and extrinsic stimuli, as well as developmental signals. Senescent cells remain viable, have alterations in metabolic activity and undergo dramatic changes in gene expression and develop a complex senescence-associated secretory phenotype. Cellular senescence can compromise tissue repair and regeneration, thereby contributing toward aging, and the number of senescent cells increases with aging. Removal of senescent cells can attenuate age-related tissue dysfunction and extend health span. However, the primary purpose of senescence is to prevent the propagation of damaged cells and to trigger their demise by the immune system. Accordingly, senescence can act as a potent anti-tumor mechanism, by preventing proliferation of potentially cancerous cells. It is a cellular program which acts as a double-edged sword, with both beneficial and detrimental effects on the health of the organism. As a cellular checkpoint, however, it requires an efficient cell replacement system that involves clearance of senescent cells and mobilization of progenitors to re-establish cell numbers. In aged organisms, this turnover system may become inefficient or may exhaust the regenerative capacity of progenitor cells, eventually resulting in the accumulation of senescent cells that may aggravate the damage and contribute to aging.

### Telomere Shortening

Telomere shortening is a consequence of the end replication problem and occurs in somatic cells without telomerase activity. Telomere shortening is one of the more widely accepted theories of aging within the scientific community.

### Deregulated Nutrient Sensing (Altered Metabolism)

Nutrient sensing pathways are commonly deregulated in human metabolic diseases. Metabolic activities can put stress on our cells. Too much activity, and changes in nutrient availability and composition cause cells to age faster. Consistent with the relevance of deregulated nutrient-sensing as a hallmark of aging, dietary restriction (DR) has been shown to increase lifespan or healthspan in all investigated eukaryote species. Current available evidence supports the idea that anabolic signaling accelerates aging, and decreased nutrient signaling extends longevity.

Even more, a pharmacological manipulation that mimics a state of limited nutrient availability, such as rapamycin, has been shown to extend longevity in mice.

### Altered Cell-Cell Communication

Aging involves changes at the level of intercellular communication, wherein the changes can be endocrine, neuroendocrine or neuronal. One of the most prominent changes in cell signaling biomarkers is “inflammaging”, the development of a chronic low-grade inflammation throughout the body with advanced age. The normal role of inflammation is to recruit the body's immune system and repair mechanisms to a specific damaged area for as long as the damage and threat are present. The constant presence of inflammation markers throughout the body wears out the immune system and damages healthy tissue. It has been shown that neurohormonal signaling (e.g., renin-angiotensin, adrenergic, insulin-IGF1 signaling) tends to be deregulated in aging as inflammatory reactions increase, immunosurveillance against pathogens and premalignant cells declines, and the composition of the peri- and extracellular environment changes, thereby affecting the mechanical and functional properties of all tissues.

### Stem Cell Exhaustion

Stem cells are undifferentiated or partially differentiated cells that can proliferate indefinitely. For the first few days after fertilization, the embryo consists almost entirely of stem cells. As the fetus grows, the cells multiply, differentiate and assume their appropriate function within the organism. In adults, stem cells are mostly located in areas that undergo gradual wear (intestine, lung, mucosa, skin) or need continuous replenishment (red blood cells, immune cells, sperm cells, hair follicles).

Loss of regenerative ability is one of the most obvious consequences of aging. This is largely because the proportion of stem cells and the speed of their division gradually lowers over time. It has been found that stem cell rejuvenation can reverse some of the effects of aging at the organismal level.

### Models for Studying Aging

The nematode Caenorhabditis elegans (C. elegans) is a nematode worm and is significantly anatomically simpler than a human, however, it does share many similarities at the molecular level making it a good candidate for a model organism. C. elegans is regarded as a great organism to study aging as most pathways affecting the aging processes are conserved between the nematode and humans¹¹. Importantly, C. elegans also allows us to study the mechanisms of how the aging process leads to development of age-related diseases.

### Effects of Krill Derived Phospholipid Compositions

Previous studies using krill oil as a supplement have shown to improve motor abnormalities and cognitive deficits in an MS (Multiple sclerosis) mouse model¹⁰. Further it has been demonstrated that LPC-bound EPA/DHA effectively enhances DHA availability in the brain and improves brain function⁶, making it a unique nutraceutical for treating AD brain⁹. Furthermore, phospholipid bound DHA and EPA has been shown to ameliorate impairment of Aβ amyloid aggregation via anti-inflammatory, antioxidative properties of krill oil 8. In an MPTP-induced PD mouse model, astaxanthin protects dopaminergic neurons in the nigro-striatal circuit in young mice, but not in older animals⁴⁰. However, up until now there has been no evidence that krill derived products can influence cellular processes that contribute to aging, or that such products have potential in prophylactic or therapeutic treatment of age-related damages and diseases.

In Example 1, C. elegans is used to explore whether a krill derived composition comprising phospholipid-bound EPA and DHA may promote healthy aging. To evaluate the potential to attenuate age-related damages and diseases, Parkinson's disease (PD) is used as a model, because aging is the major risk factor for this devastating neurodegenerative disease (NDDs). Transgenic strains expressing human α-synuclein ((t-SYN) tagged with green fluorescent protein (GFP in dopaminergic neurons)¹²⁻⁵ were used in the experiments. These transgenic strains, facilitates visualization of the age-related degeneration of dopaminergic neurons and aggregation of α-SYN aggregates in the body muscles; the two cardinal pathologic features of PD.

The results of Example 1 demonstrated that the krill derived phospholipid composition counteracts the primary drivers of aging, including oxidative stress, proteotoxic stress, mitochondrial dysfunction, senescence, and genomic instability in C. elegans and in human fibroblasts. In the C. elegans model of Parkinson's disease, krill derived phospholipid composition protects dopaminergic neurons from aging-related degeneration, decreases alpha synuclein aggregation, and improves dopamine-dependent behavior and cognition. Mechanistically, krill derived phospholipid composition increases neuronal resilience through temporal transcriptome rewiring to promote anti-oxidative stress and inflammation via healthspan regulating transcription factors such as SNK-1. However, the phospholipid compositions also promotes DA neuron survival through regulation of synaptic transmission and neuronal functions via PBO-2 and RIM-1. Collectively, krill derived phospholipid composition rewires global gene expression programs and promotes healthy aging via abrogating multiple ageing hallmarks.

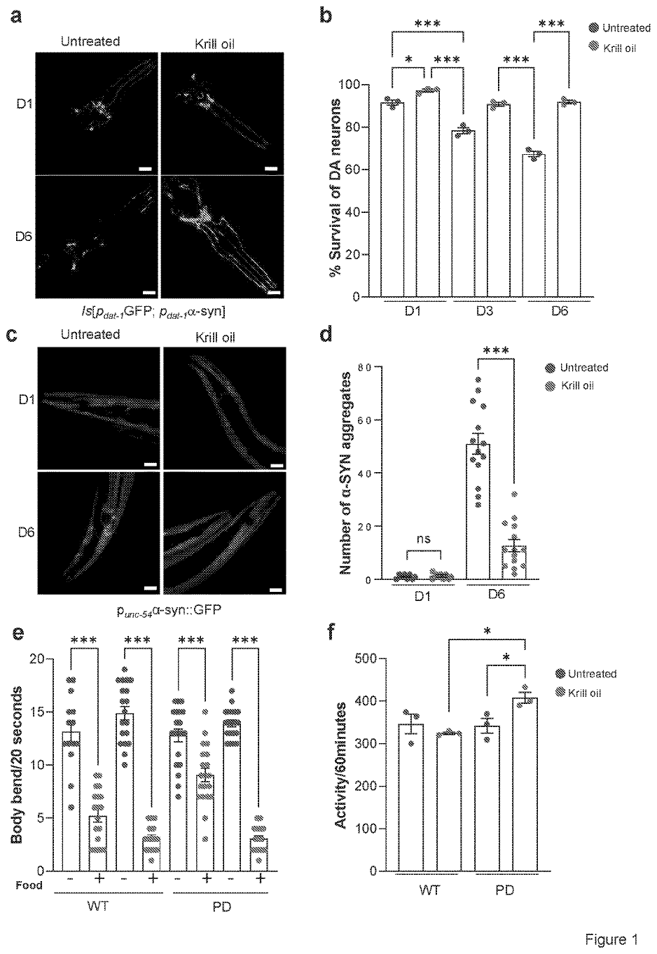

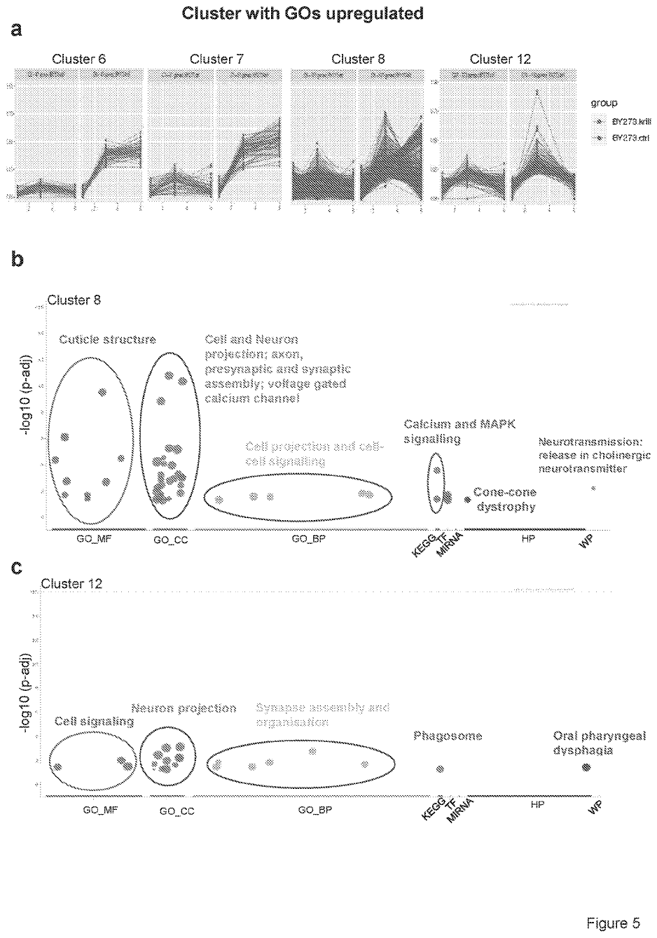

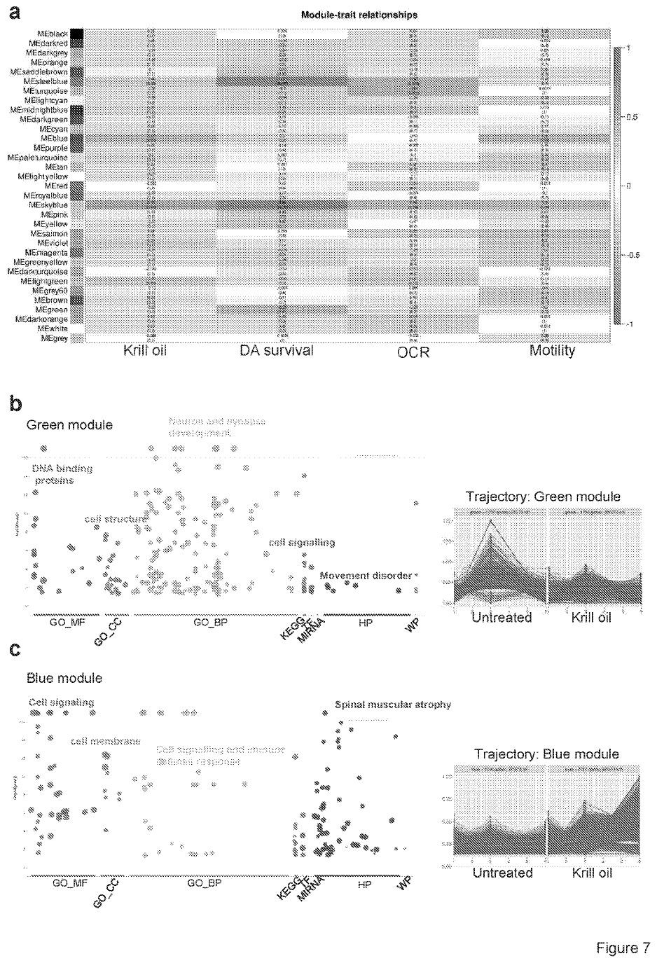

It is demonstrated that the krill derived phospholipid compositions can effectively protect dopaminergic neurons and reduce α-synuclein aggregation in aging PD worms (FIG. 1a-d). This data directly correlates to the RNAseq analysis derived clusters 4, 6, 7, 8 and 12, which represent most of the upregulated genes in the PD animals treated with the krill derived compositions (FIG. 5) and the green module from WGCNA (FIG. 7). The enriched GOs in cluster C5 are linked to cellular component morphogenesis, metalloendopeptidase activity. A recent study confirmed that these metallopeptidases play a role in age-related muscular degeneration⁴¹. This suggests the possibility that the krill derived phospholipid compositions might sustain mobility and BSR in old PD animals through upregulation of metallopeptidase activity.

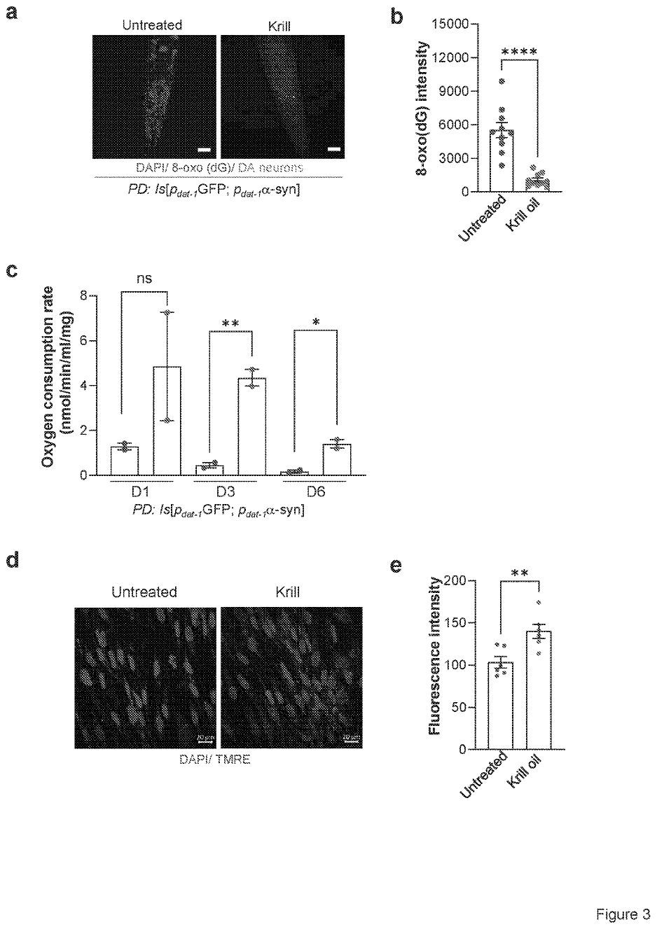

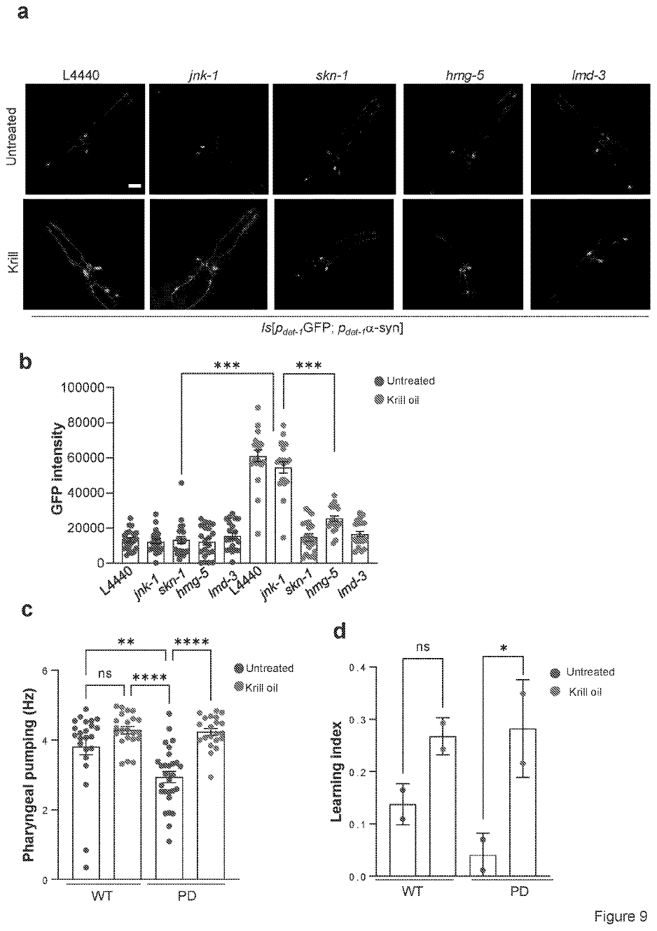

It has been reported that the aging mechanism may be influenced by gender. In males, mitochondrial gene expression declines with age⁴². The elevated expression of GOs in cluster C10 related to sex determination and reproductive organ development in response to krill derived phospholipid composition can be attributed to improved mitochondrial health. It was observed that the krill derived phospholipid composition improves mitochondrial health (FIG. 3) and elevates expression of the mitochondrially encoded genes ctc-1 and nduo-2. Notably, the master regulator of mitochondrial transcription HMG-5 is also involved in the induced neuroprotection of DA neurons. This clearly suggest that the krill derived phospholipid composition improves mitochondria health by improving mitochondria transcription activation and promote DA neuron protection. Mitochondria also plays an important role in regulating oxidative stress. With the reverse genetic approach, it is shown that the protection of DA neurons in response to krill derived phospholipid compositions depend on oxidative stress regulators skn-1 and lmd-3 (FIGS. 9a and 9b).

It is demonstrated that the PL-composition derived from krill tested in Example 1 improves dopamine dependent behavior like BSR and pharyngeal pumping in old PD animals (FIG. 1e and FIG. 9c). Interestingly, GOs for synapse organization are found in the 122-gene cluster C12 (FIGS. 5a and 5c). It is possible therefore that the test composition improves pharyngeal pumping and motoric activity in the PD strain due to better synapse organization. Moreover, the C. elegans PD model further confirmed that neuroprotection of DA neurons clearly depended on cnnm-3, pbo-2 and rim-1. This substantiates that the tested PL-composition improves the dopamine receptor signaling pathway, positive regulation of acetylcholine secretion, cholinergic synaptic transmission signaling and transmembrane transporter activity via vertebrate CNNM family homolog.

The downregulation of certain GOs in cluster 16, 22, 30 and 31 in response to the tested PL-composition is interesting; C16 represents heme and iron-ion binding proteins. Heme and iron-ion binding proteins have been linked to the early onset of Alzheimer's disease 43. Consistently, a C. elegans AD strain displayed improved memory in day 1 old adults fed with the krill derived phospholipid compositions. Similarly, overexpression of the pan-neuronal Tau protein in the C. elegans AD model BR5270 strain resulted in a 60% induction of senescence at day 5. The AD animals fed with the krill derived PL-compositions showed a 30 percent reduction in positive β-gal staining. Cluster 31 predicted downregulation of GOs in pantothenate biosynthesis and rode-cone cell atrophy (HP: human phenotype). Pantothenate kinase-associated neurodegeneration (PKAN) is exacerbated by iron buildup in the basal ganglia, resulting in clinical symptoms such as Parkinson's disease and retinal degeneration⁴⁴. This might suggest that the neuroprotection of DA neurons in PD animals fed with krill derived PL-compositions may also be affected by downregulation of pantothenate biosynthesis pathway. The observation that krill derived PL-composition downregulated genes associated with rode-cone cell atrophy, suggest that the claimed compositions could also be a useful supplement for treating retinal degeneration. In krill derived PL-composition-fed PD animals, the expression of UDP-glucuronosylthransferase is reduced in clusters 22 and 31. In early development, UDP-glucuronosylthransferase is highly upregulated and governs hormonal signaling. This may be an indication that the claimed PL-composition also can influence aging by modulating the liver enzyme UDP-glucuronosylthransferase expression 3⁷.

### CONCLUSION

The study in Example 1 demonstrates that the krill derived phospholipid composition efficiently improves dopaminergic neuron survival by improving the clearance of α-synuclein aggregation. Accordingly, the health benefit of the claimed phospholipid composition is demonstrated by the improvement of mitochondrial health, delaying senescence, maintaining genomic stability and improving proteostasis (FIG. 9). From the RNAseq data analysis it is confirmed that the tested compositions improve the cholinergic, dopaminergic and transmembrane transport system. Thus, the antioxidant and anti-inflammatory therapies such as supplementation with the described phospholipid compositions might serve as a new possible approach for healthy brain aging interventions. Based on the results it is concluded that the krill derived phospholipid composition modulated separate gene expression programs in mid-life and in old animals that both promote healthy aging by attenuating several hallmarks of aging, proteotoxic stress and senescence and promotes genomic stability and mitochondrial health. Together, these results demonstrate remarkable protection of DA neuron survival in aging animals.

Accordingly, the invention provides marine derived phospholipid compositions and methods for ameliorating several hallmarks of aging and age-related damages. The compositions as described herein are demonstrated to counteract primary drivers of aging, such as mitochondrial dysfunction, senescence, and genomic instability. Accordingly, the effect of the described phospholipid compositions as a longevity supplement are demonstrated.

In a first aspect, it is provided phospholipid compositions and methods for ameliorating at least one aging hallmark in a subject comprising administering to the subject the present composition, wherein the aging hallmark is selected from the group consisting of such as genomic instability, telomere attrition, epigenetic alterations, loss of proteostasis, deregulated nutrient sensing, mitochondrial dysfunction, cellular senescence, stem cell exhaustion, and altered intercellular communication.

In one embodiment, the present method ameliorates genomic instability. In another embodiment, the present method ameliorates telomere attrition. In another embodiment, the present method ameliorates one or more epigenetic alterations. In another embodiment, the present method ameliorates loss of proteostasis. In another embodiment, the present method ameliorates deregulated nutrient sensing. In another embodiment, the present method ameliorates mitochondrial dysfunction. In another embodiment, the present method ameliorates cellular senescence. In another embodiment, the present method ameliorates stem cell exhaustion. In another embodiment, the present method ameliorates altered intercellular communication.

### Phospholipid Compositions

The present invention provides marine derived phospholipid compositions and their use in therapy, pharmaceuticals, nutraceuticals, medical foods and functional foods.

Normal intake of PLs is approximately 2-8 grams per day, and this represent 1-10% of dietary fat intake. Phosphatidylcholine (PC) is an important phospholipid as it contains choline which is important in many body functions, such as nerve signaling and liver and muscle function. Choline is an essential nutrient that is crucial for normal cellular function and is particularly important for liver health, heart health and brain development.

PLs are present in eggs, milk, meat and fish, however, the common fatty acids in these PLs are palmitic, stearic, oleic and linoleic acid. Many marine derived phospholipids are unique due to high levels of EPA and DHA. In krill as much as 80% or higher of the total amount of EPA and DHA can be bound to phospholipids. In addition, a predominant part of the EPA and DHA found in krill oil is bound to PC. In contrast, EPA and DHA in fish oil are mostly bound to triglycerides (TGs).

The metabolism of omega-3s (EPA/DHA) is highly important in evaluating bioavailability and retention in the body.

In preferred embodiments, the phospholipid compositions are prepared from krill, for example Euphausia superba or Euphausia pacifica. In other preferred embodiments, the phospholipid compositions are prepared from fish or fish by-products (e.g., herring, herring roe, salmon, anchovy, mackerel and associated by-products from processing the fish) Calanus, shrimp, molluscs (including cephalopods such as squid), or algae. Lipid compositions from herring, herring roe, squid and some microalgae (such as f ex some strains of Chlorella) are shown to be rich in phospholipids, including phosphatidylcholine. Suitable krill crude phospholipid extracts, desalted krill phospholipid extracts and methods for processing krill are disclosed in PCT/IB2016/000208 and PCT/IB2016/000326, each of which is incorporated by reference herein in its entirety.

In preferred embodiments, the phospholipid compositions of the present invention are derived from krill.

In preferred embodiments of the invention, the phospholipid composition comprises phospholipid bound EPA and/or phospholipid bound DHA. The amount of phospholipid bound EPA and/or DHA may preferably be determined by methods known in the art, including by 31P-NMR procedures. When determining the amount of EPA and/or DHA present in a composition that is bound to phospholipids and where an NMR method is utilized, the amount of bound EPA and/or DHA may be presented as a weight percent relative to the total weight of the composition i.e., w/w. While it is contemplated that the majority of EPA and/or DHA in the compositions will be bound to phospholipid molecules in the composition, some EPA and/or DHA may also be bound to other molecules such as triglycerides. In some preferred embodiments, the total amount of EPA and/or DHA in a composition (whether bound to phospholipids or triglycerides or present as free fatty acids, for example) may be determined by GC-FAME and expressed as g/100 g fatty acids. Alternatively, the total amount of EPA and/or DHA in a mixed composition may also be expressed as a weight percent based on the total weight of the composition, i.e., w/w.

Accordingly, in some preferred embodiments, at least 50% on a molar basis of the EPA and/or DHA present in a composition comprising phospholipid bound EPA and/or DHA is bound to a phospholipid molecule, including lyso-forms. In some preferred embodiments, at least 60% on a molar basis of the EPA and/or DHA present in a composition comprising phospholipid bound EPA and/or DHA is bound to a phospholipid molecule, including lyso-forms. In some preferred embodiments, at least 70% on a molar basis of the EPA and/or DHA present in a composition comprising phospholipid bound EPA and/or DHA is bound to a phospholipid molecule, including lyso-forms. In some preferred embodiments, at least 80% on a molar basis of the EPA and/or DHA present in a composition comprising phospholipid bound EPA and/or DHA is bound to a phospholipid molecule, including lyso-forms. In some preferred embodiments, at least 90% on a molar basis of the EPA and/or DHA present in a composition comprising phospholipid bound EPA and/or DHA is bound to a phospholipid molecule, including lyso-forms. In these embodiments, the amount of phospholipid bound EPA and/or DHA relative to other molecular species containing EPA and/or DHA may preferably be determined by 31P-NMR.

In some preferred embodiments, the phospholipid composition comprising phospholipid bound EPA and/or DHA may be further characterized by the total amount of EPA and/or DHA in the composition represented as grams of EPA and/or DHA per 100 grams of fatty acids as determined by GC-FAME. Accordingly, in some preferred embodiments, the amount of EPA and/or DHA in the composition is from 5 to 80 g/100 g fatty acids. In some preferred embodiments, the amount of EPA and/or DHA in the composition is from 5 to 60 g/100 g fatty acids. In some preferred embodiments, the amount of EPA and/or DHA is from 5 to 50 g/100 g fatty acids. In some preferred embodiments, the amount of bound EPA and/or DHA in the composition is from 5 to 40 g/100 g fatty acids. In some preferred embodiments, the amount of EPA and/or DHA in the composition is from 10 to 40 g/100 g fatty acids. In some preferred embodiments, the amount of EPA and/or DHA in the composition is from 10 to 30 g/100 g fatty acids. In some preferred embodiments, the amount of EPA and/or DHA in the composition is from 15 to 40 g/100 g fatty acids. In some preferred embodiments, the amount of bound EPA and/or DHA in the composition is from 15 to 30 g/100 g fatty acids.

In further preferred embodiments, the phospholipid composition comprising phospholipid bound EPA and/or DHA may be further characterized by the total amount of EPA and/or DHA in the composition represented as weight percent of the EPA and/or DHA as per the total weight of the composition. Accordingly, in some preferred embodiments, the phospholipid composition comprising phospholipid bound EPA and/or DHA comprises from 10% to 70% EPA and/or DHA w/w (i.e., grams of EPA and/or DHA whether bound or free per the total weight of the composition). In some preferred embodiments, the phospholipid composition comprising phospholipid bound EPA and/or DHA comprises from 10% to 50% EPA and/or DHA w/w. In some preferred embodiments, the phospholipid composition comprising phospholipid bound EPA and/or DHA comprises from 10% to 40% EPA and/or DHA w/w. In some preferred embodiments, the phospholipid composition comprising phospholipid bound EPA and/or DHA comprises from 20% to 60% EPA and/or DHA w/w. In some preferred embodiments, the phospholipid composition comprising phospholipid bound EPA and/or DHA comprises from 20% to 50% EPA and/or DHA w/w. In some preferred embodiments, the phospholipid composition comprising phospholipid bound EPA and/or DHA comprises from 20% to 40% EPA and/or DHA w/w.

In further preferred embodiments, the phospholipid composition comprising phospholipid bound EPA and/or DHA may be further characterized by the ratio of EPA and/or DHA present in the composition. Accordingly, in some preferred embodiments, the ratio of EPA to DHA in the composition is from 1:1 to 5:1. In some preferred embodiments, the ratio of EPA to DHA in the composition is from 1:1 to 3:1. In some preferred embodiments, the ratio of EPA to DHA in the composition is from 1:1 to 2.5:1. In some preferred embodiments, the ratio of DHA to EPA in the composition is from 1:1 to 5:1. In some preferred embodiments, the ratio of DHA to EPA in the composition is from 1:1 to 3:1. In some preferred embodiments, the ratio of DHA to EPA in the composition is from 1:1 to 2.5:1.

In further preferred embodiments, the phospholipid composition comprising phospholipid bound EPA and/or DHA may be further characterized by the total amount of phospholipids in the composition. In some preferred embodiments, the amount of total phospholipids, including lyso-forms is determined by 31P-NMR to provide the total on a w/w basis (weight per total weight of the composition). In some preferred embodiments, the compositions comprise from 10% to 90% total phospholipids (w/w). In some preferred embodiments, the compositions comprise from 10% to 80% total phospholipids (w/w). In some preferred embodiments, the compositions comprise from 10% to 70% total phospholipids (w/w). In some preferred embodiments, the compositions comprise from 10% to 60% total phospholipids (w/w). In some preferred embodiments, the compositions comprise from 20% to 90% total phospholipids (w/w). In some preferred embodiments, the compositions comprise from 20% to 80% total phospholipids (w/w). In some preferred embodiments, the compositions comprise from 20% to 70% total phospholipids (w/w). In some preferred embodiments, the compositions comprise from 20% to 60% total phospholipids (w/w). In some preferred embodiments, the compositions comprise from 30% to 90% total phospholipids (w/w). In some preferred embodiments, the compositions comprise from 30% to 80% total phospholipids (w/w). In some preferred embodiments, the compositions comprise from 30% to 70% total phospholipids (w/w). In some preferred embodiments, the compositions comprise from 30% to 60% total phospholipids (w/w).

In some embodiments, the phospholipid composition comprising phospholipid bound EPA and/or DHA may be further characterized by the total amount of other lipids in the composition. In some embodiments the composition comprises about 26% w/w to 46% w/w triglycerides, more preferably from about 31% w/w to about 41% w/w triglycerides, and most preferably about 36% w/w triglycerides, wherein w/w refers to the weight of the triglycerides as a percent of the total phospholipid composition weight. In some embodiments, the phospholipid composition preferably comprises from about 1% w/w to 7% w/w diglycerides, more preferably from about 1.5% w/w to about 4.5% w/w diglycerides, and most preferably about 3% w/w diglycerides, wherein w/w refers to the weight of the diglycerides as a percent of the total phospholipid composition weight. In some further embodiments, the phospholipid composition preferably comprises from about 2% w/w to 12% w/w free fatty acids, more preferably from about 4.5% w/w to about 9.5% w/w free fatty acids, and most preferably about 7% w/w free fatty acids, wherein w/w refers to the weight of the free fatty acids as a percent of the total phospholipid composition weight. In some embodiments, the phospholipid composition preferably comprises from about 1% w/w to 7% w/w cholesterol, more preferably from about 1.5% w/w to about 4.5% w/w cholesterol, and most preferably about 3% w/w cholesterol, wherein w/w refers to the weight of the cholesterol as a percent of the phospholipid composition weight. In some embodiments, the krill phospholipid composition preferably comprises from about 0.1% w/w to 2% w/w cholesterol esters, more preferably from about 0.2% w/w to about 1.0% w/w cholesterol esters, and most preferably about 0.5% w/w cholesterol esters, wherein w/w refers to the weight of the cholesterol esters as a percent of the phospholipid composition weight. In one embodiment, the phospholipid composition comprises a predominant amount of phosphatidylcholine (PC) compared to an amount of lyso-phosphatidylcholine (LPC). Accordingly, in some preferred embodiments, the compositions can be further characterized by the PC to LPC ratio. In some preferred embodiments, the ratio of PC to LPC in the composition is from 2:1 to 50:1. In some preferred embodiments, the ratio of PC to LPC in the composition is from 5:1 to 50:1. In some preferred embodiments, the ratio of PC to LPC in the composition is from 5:1 to 20:1.

In embodiments, where the compositions comprise a predominant amount of PC, the compositions may be further characterized based on the total amount of PC on a w/w basis in the composition, which may preferably be determined by 31P-NMR. In some preferred embodiments, the compositions comprise from 20% to 70% PC (w/w). In some preferred embodiments, the compositions comprise from 20% to 60% PC (w/w). In some preferred embodiments, the compositions comprise from 20% to 50% PC (w/w). In some preferred embodiments, the compositions comprise from 30% to 70% PC (w/w). In some preferred embodiments, the compositions comprise from 30% to 60% PC (w/w). In some preferred embodiments, the compositions comprise from 30% to 50% PC (w/w).

In another embodiment, the phospholipid composition comprises a predominant amount of an LPC-compound compared to an amount of PC. LPC compositions can be made by enzymatic treatment of the phospholipid extract. Methods for processing phospholipids into lyso-phospholipids are disclosed in PCT/IB2018/001588.

Accordingly, in some preferred embodiments, the compositions can be further characterized by the LPC to PC ratio. In some preferred embodiments, the ratio of LPC to PC in the composition is from 1:1 to 20:1. In some preferred embodiments, the ratio of LPC to PC in the composition is from 1:1 to 10:1. In some preferred embodiments, the ratio of LPC to PC in the composition is from 1:1 to 5:1. In some preferred embodiments, the ratio of LPC to PC in the composition is from 2:1 to 5:1.

In embodiments, where the compositions comprise a predominant amount of LPC, the compositions may be further characterized based on the total amount of LPC on a w/w basis in the composition, which may preferably be determined by 31P-NMR. In some preferred embodiments, the compositions comprise from 10% to 70% LPC (w/w). In some preferred embodiments, the compositions comprise from 10% to 60% LPC (w/w). In some preferred embodiments, the compositions comprise from 10% to 50% LPC (w/w). In some preferred embodiments, the compositions comprise from 15% to 50% LPC (w/w). In some preferred embodiments, the compositions comprise from 15% to 40% LPC (w/w). In some preferred embodiments, the compositions comprise from 15% to 30% LPC (w/w).

Both krill oil (Superba Boost) and further processed krill derived LPC-compositions (Lysoveta) are rich in phospholipid-bound EPA and DHA, as well as astaxanthin and choline.

Accordingly, in some preferred embodiments, the compositions can be further characterized by the amount of choline on a w/w basis in the composition, which may preferably be determined by 31P-NMR. In some preferred embodiments, the compositions comprise from 2% to 20% choline (w/w). In some preferred embodiments, the compositions comprise from 2% to 15% choline (w/w). In some preferred embodiments, the compositions comprise from 2% to 10% choline (w/w). In some preferred embodiments, the compositions comprise from 5% to 20% choline (w/w). In some preferred embodiments, the compositions comprise from 5% to 15% choline (w/w). In some preferred embodiments, the compositions comprise from 5% to 10% choline (w/w).

In further embodiments, the phospholipid composition comprising phospholipid bound EPA and/or DHA may be further characterized by the total amount of phosphatidylcholine. In some preferred embodiments, the composition comprising phospholipids having bound EPA and/or DHA is characterized in having a phosphatidylcholine (PC) content of from 20 to 70%, 20 to 60%, or 20 to 50% w/w, where w/w is determined by dividing the total weight of PC molecules with two fatty acids attached by the total weight of the composition and may preferably be determined by 31P-NMR. In other embodiments, the amount of PC may be expressed w/w of total phospholipids.

In other embodiments, the amount of PC may be expressed w/w of total phospholipids. In these embodiments the composition may be characterized in comprising about 75% w/w to 95% w/w phosphatidylcholine, more preferably from about 80% w/w to about 90% w/w phosphatidylcholine, and most preferably about 85% to 90% w/w phosphatidylcholine, wherein w/w refers to the weight of the phosphatidylcholine as a percent of the total phospholipid weight.

In some preferred embodiments, the compositions can be further characterized by the amount of astaxanthin esters in mg/kg of the composition, preferably measured by UV spectroscopy. In some preferred embodiments, the composition comprises from 50 to 1000 mg/kg astaxanthin esters. In some preferred embodiments, the composition comprises from 50 to 500 mg/kg astaxanthin esters.

In some preferred embodiments, the composition comprises from 50 to 300 mg/kg astaxanthin esters. In some preferred embodiments, the composition comprises from 100 to 400 mg/kg astaxanthin esters. In some preferred embodiments, the composition comprises from 100 to 300 mg/kg astaxanthin esters.

In some preferred embodiments, the compositions can be further characterized by the amount of TMAO in the composition, preferably expressed as mgN/100 g. In some preferred embodiments, the amount of TMAO is from 1 to 30 mgN/100 g. In some preferred embodiments, the amount of TMAO is from 1 to 20 mgN/100 g. In some preferred embodiments, the amount of TMAO is from 1 to 10 mgN/100 g.

In some preferred embodiments, the compositions can be further characterized by their peroxide value (PV), preferably determined by the acetic acid-isooctane method. In some preferred embodiments, the peroxide value is less than 10. In some preferred embodiments, the peroxide value is less than 5. In some preferred embodiments, the peroxide value is less than 3. In some preferred embodiments, the peroxide value is less than 2. In some preferred embodiments, the peroxide value is less than 5.

In some preferred embodiments, the compositions can be further characterized by their acid value, preferably determined by AOCS Cd 3d-63. In some preferred embodiments, the acid value is less than 50. In some preferred embodiments, the acid value is less than 40. In some preferred embodiments, the acid value is less than 30. In some preferred embodiments, the acid value is less than 20.

In some preferred embodiments, the compositions can be further characterized by the Na⁺ content, preferably expressed on a w/w basis per the total weight of the composition. In some preferred embodiments, the compositions comprise less than 1.000% Na⁺ (w/w). In some preferred embodiments, the compositions comprise less than 0.500% Na⁺ (w/w). In some preferred embodiments, the compositions comprise less than 0.300% Na⁺ (w/w). In some preferred embodiments, the compositions comprise less than 0.200% Na⁺ (w/w).

In some preferred embodiments, the composition is characterised in:
i) comprising from 10 to 40 g/100 g EPA and DPA together, as assayed by gas chromatography fatty acid methylester (GC-FAME) analysis;
ii) having a ratio of EPA to DHA of from 1:1 to 3:1;
iii) having a total phospholipid content of from 20 to 60% w/w, where w/w is determined by measuring the total weight of phospholipids in the composition by 31P-NMR and dividing the total weight of the phospholipid by the total weight of the composition;
iv) having a choline content of from 2 to 10% w/w, where w/w is determined by measuring the total weight of choline in the composition by 31P-NMR and dividing the total weight of the phospholipid by the total weight of the composition;
v) having a phosphatidylcholine (PC) content of from 20 to 50% w/w, where w/w is determined by measuring the total weight of PC molecules with two fatty acids attached in the composition and dividing the total weight of PC molecules with two fatty acids attached by the total weight of the composition, optionally wherein the ratio of PC to LPC in the composition is from 2:1 to 50:1;
vi) having a lysophosphatidylcholine (LPC) content of less than 10% w/w, where w/w is determined by measuring the total weight of LPC molecules with one fatty acid attached in the composition and dividing the total weight of LPC molecules with one fatty acid attached by the total weight of the composition;
vii) comprising astaxanthin esters in an amount of from 100 to 300 mg/kg;
viii) having a TMAO content, expressed as mgN/100 g, of from 1 to 10 mgN/100 g;
ix) having a peroxide value of less than 2;
x) having an acid value of less than 30; and/or
xi) having a Na+ content of less than 0.200% w/w of the composition, where w/w is determined by measuring the total weight of Na+ in the composition and dividing the total weight of Na+ by the total weight of the composition;

In some preferred embodiments, the composition is characterised in:
i) comprising from 10 to 40 g/100 g EPA and DPA together, as assayed by gas chromatography fatty acid methylester (GC-FAME) analysis;
ii) having a ratio of EPA to DHA of from 1:1 to 3:1;
iii) having a total phospholipid content of from 20 to 60% w/w, where w/w is determined by measuring the total weight of phospholipids in the composition by 31P-NMR and dividing the total weight of the phospholipid by the total weight of the composition;
iv) having a choline content of from 2 to 10% w/w, where w/w is determined by measuring the total weight of choline in the composition by 31P-NMR and dividing the total weight of the phospholipid by the total weight of the composition;
v) having a phosphatidylcholine (PC) content of less than 20% w/w, where w/w is determined by measuring the total weight of PC molecules with two fatty acids attached in the composition and dividing the total weight of PC molecules with two fatty acids attached by the total weight of the composition;
vi) having a lysophosphatidylcholine (LPC) content of from 20 to 50% w/w, where w/w is determined by measuring the total weight of LPC molecules with one fatty acid attached in the composition and dividing the total weight of LPC molecules with one fatty acid attached by the total weight of the composition, optionally wherein the ratio of LPC to PC in the composition is from 1:1 to 20:1
vii) comprising astaxanthin esters in an amount of from 100 to 300 mg/kg;
viii) having a TMAO content, expressed as mgN/100 g, of from 1 to 10 mgN/100 g;
ix) having a peroxide value of less than 2;
x) having an acid value of less than 30; and/or
xi) having a Na+ content of less than 0.200% w/w of the composition, where w/w is determined by measuring the total weight of Na+ in the composition and dividing the total weight of Na+ by the total weight of the composition;

In some preferred embodiments, the composition is characterised in any one of i) to xi) above. In some preferred embodiments, the composition is characterised in any two of i) to xi) above. In some preferred embodiments, the composition is characterised in any three of i) to xi) above. In some preferred embodiments, the composition is characterised in any four of i) to xi) above. In some preferred embodiments, the composition is characterised in any five of i) to xi) above. In some preferred embodiments, the composition is characterised in any six of i) to xi) above. In some preferred embodiments, the composition is characterised in any seven of i) to xi) above. In some preferred embodiments, the composition is characterised in any eight of i) to xi) above. In some preferred embodiments, the composition is characterised in any nine of i) to xi) above. In some preferred embodiments, the composition is characterised in any ten of i) to xi) above. In some preferred embodiments, the composition is characterised in all of i) to xi) above.

In some preferred embodiments, the composition is characterised in i) and ii) above. In some preferred embodiments, the composition is characterised in i) and iii) above. In some preferred embodiments, the composition is characterised in i) and iv) above. In some preferred embodiments, the composition is characterised in i) and v) above. In some preferred embodiments, the composition is characterised in i) and vi) above. In some preferred embodiments, the composition is characterised in i) and vii) above. In some preferred embodiments, the composition is characterised in i) and viii) above. In some preferred embodiments, the composition is characterised in i) and ix) above. In some preferred embodiments, the composition is characterised in i) and x) above. In some preferred embodiments, the composition is characterised in i) and xi) above.

In some preferred embodiments, the composition is characterised in ii) and iii) above. In some preferred embodiments, the composition is characterised in ii) and iv) above. In some preferred embodiments, the composition is characterised in ii) and v) above. In some preferred embodiments, the composition is characterised in ii) and vi) above. In some preferred embodiments, the composition is characterised in ii) and vii) above. In some preferred embodiments, the composition is characterised in ii) and viii) above. In some preferred embodiments, the composition is characterised in ii) and ix) above. In some preferred embodiments, the composition is characterised in ii) and x) above. In some preferred embodiments, the composition is characterised in ii) and xi) above.

In some preferred embodiments, the composition is characterised in iii) and iv) above. In some preferred embodiments, the composition is characterised in iii) and v) above. In some preferred embodiments, the composition is characterised in iii) and vi) above. In some preferred embodiments, the composition is characterised in iii) and vii) above. In some preferred embodiments, the composition is characterised in iii) and viii) above. In some preferred embodiments, the composition is characterised in iii) and ix) above. In some preferred embodiments, the composition is characterised in iii) and x) above. In some preferred embodiments, the composition is characterised in iii) and xi) above.

In some preferred embodiments, the composition is characterised in iv) and v) above. In some preferred embodiments, the composition is characterised in iv) and vi) above. In some preferred embodiments, the composition is characterised in iv) and vii) above. In some preferred embodiments, the composition is characterised in iv) and viii) above. In some preferred embodiments, the composition is characterised in iv) and ix) above. In some preferred embodiments, the composition is characterised in iv) and x) above. In some preferred embodiments, the composition is characterised in iv) and xi) above.

In some preferred embodiments, the composition is characterised in v) and vi) above. In some preferred embodiments, the composition is characterised in v) and vii) above. In some preferred embodiments, the composition is characterised in v) and viii) above. In some preferred embodiments, the composition is characterised in v) and ix) above. In some preferred embodiments, the composition is characterised in v) and x) above. In some preferred embodiments, the composition is characterised in v) and xi) above.

In some preferred embodiments, the composition is characterised in vi) and vii) above. In some preferred embodiments, the composition is characterised in vi) and viii) above. In some preferred embodiments, the composition is characterised in vi) and ix) above. In some preferred embodiments, the composition is characterised in vi) and x) above. In some preferred embodiments, the composition is characterised in vi) and xi) above.

In some preferred embodiments, the composition is characterised in vii) and viii) above. In some preferred embodiments, the composition is characterised in vii) and ix) above. In some preferred embodiments, the composition is characterised in vii) and x) above. In some preferred embodiments, the composition is characterised in vii) and xi) above.

In some preferred embodiments, the composition is characterised in viii) and ix) above. In some preferred embodiments, the composition is characterised in viii) and x) above. In some preferred embodiments, the composition is characterised in viii) and xi) above.

In some preferred embodiments, the composition is characterised in ix) and x) above. In some preferred embodiments, the composition is characterised in ix) and xi) above.

In some preferred embodiments, the composition is characterised in x) and xi) above.

TABLE 1
Lipid composition and fatty acid profile in two
different PL-compositions derived from krill

| Batch Size | | | Superba | |
|---|---|---|---|---|
| Parameter | Method description | Unit | Boost | Lysoveta |
| Appearance | Visual | | Red | Red |
| | | | viscous oil | viscous oil |
| Identification | GC FAME | | Conforms | Conforms |
| Viscosity at 35° C. | Rotational Viscometer | | 1937.44 | 769.4 |
| TMAO + TMAO | Ion chromatography | mgN/100 g | <2 | <6.67 |
| Na+ (g/100 g) | Ion chromatography | g/100 g | 0.137 | |
| LIPID COMPOSITION | | | | |
| Total phospholipids | 31P NMR | g/100 g | 58.54 | 43.87 |
| PC | 31P NMR | g/100 g | 47.78 | 8.37 |
| Total LPC | 31P NMR | g/100 g | 4.08 | 26.08 |
| Choline -calculated | 31P NMR | g/100 g | 7.09 | 7.08 |
| Free fatty acids | 13C NMR | g/100 g | ND | 11.5 |
| Ethyl esters | 13C NMR | g/100 g | ND | 20 |
| Acid value | AOCS Cd 3d-63 | mg KOH/g | 18 | 26 |
| Free fatty acids | AOCS Cd 3d-63 | g/100 g | 8.84 | 13.08 |
| (as oleic acid) | | | | |
| FATTY ACID PROFILE | | | | |
| total omega-3 | GC (FAME) | g/100 g | 28.45 | 29.01 |
| fatty acids | | | | |
| C 20:5 n-3 (EPA) | GC (FAME) | g/100 g | 15.63 | 15.97 |
| C 22:6 n-3 (DHA) | GC (FAME) | g/100 g | 8.03 | 8.26 |
| STABILITY INDEX | | | | |
| Peroxide value | PV value Acetic acid- | | 1.7 | <1 |
| | Isooctane Method | | | |
| ANTIOXIDANTS | | | | |
| Astaxanthin | UV spectroscopy | mg/kg | 191.9 | 177 |
| Astaxanthin | | | Conforms | |
| esterification | | | | |
| WATER AND ETHANOL | | | | |
| Water content | Karl Fischer | g/100 g | 0.78 | 0.06 |
| Water activity | Moisture probe | | 0.23 | 0.09 |
| at 25° C. | | | | |
| Ethanol content | Gas Chromatography | g/100 g | 2.18 | 0.15 |
| MICROBIOLOGY | | | | |
| Total plate count | | cfu/g | <10 | |
| Mould and Yeast | | cfu/g | <10 | <10 |
| E. Coli | | cfu/g | Negative | <10 |
| Coliform | | cfu/g | <10 | <10 |
| Salmonella | PCR | detected/not | Negative | Negative |
| | | detected in 25 g | | |
| ADDITIONAL ANALYSIS | | | | |
| Lipids (Nofima) | | | | |
| Triacylglycerol | HPLC | g/100 g | 20 | 22 |
| Diacylglycerol | HPLC | g/100 g | 1.8 | 0.9 |
| Monoacylglycerol | HPLC | g/100 g | <1 | <0.1 |
| Free fatty acids | HPLC | g/100 g | 6.4 | 9.3 |
| Cholesterol | HPLC | g/100 g | 1.7 | 2 |
| Cholesterol esters | HPLC | | <0.5 | <0.5 |
| Phosphatidyletanolamine | HPLC | g/100 g | 6.4 | 36 |
| Phosphatidylinositol | HPLC | g/100 g | <1 | <1 |
| Phosphatidylserine | HPLC | g/100 g | <1 | <1 |
| Phosphatidylcholine | HPLC | g/100 g | 56 | 18 |
| Lyso-Phosphatidylcholine | HPLC | g/100 g | 3.9 | 43 |
| Total polar lipids | HPLC | g/100 g | 66.3 | 64.6 |
| Total neutral lipids | HPLC | g/100 g | 29.9 | 34.2 |
| Total sum lipids | HPLC | g/100 g | 96.2 | 98.8 |
| Fatty acid composition | | | | |
| C12:0 | GC (FAME) | g/100 g | 0.009 | 0.08 |
| C14:0 | GC (FAME) | g/100 g | 4.74 | 4.67 |
| C15:0 | GC (FAME) | g/100 g | 0.22 | 0.23 |
| C16:0 | GC (FAME) | g/100 g | 13.94 | 13.85 |
| 16:1 n-7 | GC (FAME) | g/100 g | 3.21 | 3.21 |
| C18:0 | GC (FAME) | g/100 g | 1.31 | 1.24 |
| C18:1 n-9 | GC (FAME) | g/100 g | 5.51 | 5.6 |
| C18:1 n-7 | GC (FAME) | g/100 g | 4.04 | 4.09 |
| C18:2 n-6 | GC (FAME) | g/100 g | 1.35 | 1.38 |
| C18:3 n-3 | GC (FAME) | g/100 g | 1.03 | 1.03 |
| C18:4 n-3 | GC (FAME) | g/100 g | 2.47 | 2.49 |
| C20:1 n-9 | GC (FAME) | g/100 g | 0.35 | 0.36 |
| C20:4 n-6 | GC (FAME) | g/100 g | 0.35 | 0.35 |
| C20:3 n-3 | GC (FAME) | g/100 g | 0 | 0 |
| C20:4 n-3 | GC (FAME) | g/100 g | 0.42 | 0.42 |
| C20:5 n-3 | GC (FAME) | g/100 g | 15.63 | 15.97 |
| C22:1 n-9 | GC (FAME) | g/100 g | 0.61 | 0.62 |
| C21:5 n-3 | GC (FAME) | g/100 g | 0.46 | 0.47 |
| C22:5 n-3 | GC (FAME) | g/100 g | 0.36 | 0.37 |
| C22:6 n-3 | GC (FAME) | g/100 g | 8.08 | 8.26 |
| Total FA | GC (FAME) | g/100 g | 64.15 | 64.68 |
| SFA | GC (FAME) | g/100 g | 20.3 | 20.06 |
| MUFA | GC (FAME) | g/100 g | 13.71 | 13.87 |
| PUFA n-6 | GC (FAME) | g/100 g | 1.69 | 1.73 |
| EPA + DHA | GC (FAME) | g/100 g | 23.71 | 24.23 |
| PUFA n-3 | GC (FAME) | g/100 g | 28.45 | 29.01 |
| Total PUFA | GC (FAME) | g/100 g | 30.14 | 30.74 |
| Unknown FA | GC (FAME) | g/100 g | 3.72 | 4.08 |

NMR method according to USP/FCC

### Therapeutic Applications

1. Protect against neurodegeneration in brain.

The finding that a krill derived phospholipid composition attenuated several hallmarks of aging in C. elegans and in human fibroblasts, support that dietary krill derived phospholipid compositions prevents basic aging mechanisms in neuronal tissues. As such, the krill derived phospholipids compositions have demonstrated neuroprotective benefits, which have great implications for brain health and for treating age-related neurodegeneration and neurodegenerative diseases (NDDs) in the brain.

Although systemic diseases take the biggest toll on human health and well-being, increasingly, a failing brain is the arbiter of a death preceded by a gradual loss of the essence of being. Aging, which is fundamental to neurodegeneration and dementia, affects every organ in the body and seems to be encoded partly in a blood-based signature. Indeed, factors in the circulation have been shown to modulate aging and to rejuvenate numerous organs, including the brain. Because brain tissue is composed primarily of postmitotic cells, it is especially sensitive to the effects of aging.

Studies suggest that uptake of EPA and DHA to the brain is greater when EPA and DHA are in the form of LPC-EPA and LPC-DHA in the blood. The preferential crossing of the blood-brain barrier in the LPC-EPA/DHA form is demonstrated in several studies, which is supported by discovery of the specific transporter of LPC-EPA/DHA called Mfsd2a (major facilitating superfamily domain-containing protein 2A) (Nguyen et al 2014).This has important implications for treatments in the brain.

Based on the documentation of effects, the invention is a method for ameliorating at least one aging hallmark in a subject comprising administering to a subject in need thereof an effective amount of phospholipid-bound EPA and/or DHA compounds or a phospholipid composition as described above.

The invention may also be defined as a phospholipid-bound EPA and/or DHA compounds or a phospholipid composition as described above for ameliorating at least one hallmark of aging or for use in prophylaxis and/or therapy of an age-related neurodegenerative disorder.

In some embodiments of the invention, an effective amount of the phospholipid-bound EPA/DHA compounds, or the phospholipid compositions described above, are administered to a subject in need thereof
to treat, prevent, or improve normal age-related neurodegeneration and neurodegenerative disorders, or
to treat or prevent Alzheimer's disease or Parkinson's disease
to treat, prevent or improve decline in executive functions or age-related cognitive decline, or
   to treat, prevent, or improve cognition and/or a cognitive disease, disorder or impairment (memory, concentration, learning (deficit)). In one particular preferred embodiment the composition for use in therapy or in the methods of treatment of neuronal tissues in the brain (incl. neurodegeneration, cognitive disease, disorder or impairment or decline in executive functions) as described comprises a phospholipid-bound EPA and/or DHA wherein a predominant amount of EPA and/or DHA is bound to a LPC-compound compared to the amount of EPA and/or DHA bound to a PC

### 2. Protect Against Age-Related Cellular Damage

The finding that a krill derived phospholipid composition attenuated several hallmarks of aging such as mitochondrial dysfunction, senescence, and genomic instability in C. elegans and in human fibroblasts, support that dietary krill derived phospholipid compositions also prevents basic aging mechanisms in tissues other than neuronal tissues. In support of this, effects have been demonstrated in different organs following supplementation with krill derived phospholipid compositions. For example, significant increases in muscle size and function were found in healthy older adults following 6 months of dietary supplementation with a krill derived phospholipid composition (Alkhedhairi, S A A et al, Clinical Nutrition 41 (2022) 1228-1235). It has also been demonstrated that krill derived phospholipids fed to mice suppressed high fat diet induced inflammatory signaling pathways in adipose tissue and liver (Gart et al, Nutrients 2021, 13, 2836).

Cellular senescence has been recognized as the most important age-associated phenotypic change linked to the concept of tissue aging.⁴⁵ Historically, senescence has been viewed as an irreversible cell-cycle arrest mechanism that acts to protect against cancer, but discoveries have extended its known role to complex biological processes such as development, tissue repair, ageing and age-related disorders. During ageing-related senescence, the switch from temporal to persistent cell-cycle arrest appears unscheduled and stochastic in nature, probably involving the combined effects of distinct senescence-inducing stressors acting simultaneously on a cell.

Several studies are now focused on clarifying the specific molecular pathways of aging in different cells, tissues, or organs, and senescent cells are found to be drivers of a large number of age-related pathologies.⁴⁶ In addition to Alzheimer's and Parkinson's disease, these pathologies include atherosclerosis, cardiovascular dysfunction (including cardiovascular problems caused by certain genotoxic chemotherapies), tumor progression, loss of hematopoietic and skeletal muscle stem cell functions, non-alcoholic fatty liver disease, pulmonary fibrosis, osteoarthritis and osteoporosis. Within the liver, biliary and vascular components have emerged as important determinants of some form of liver disease.⁴⁷ Several findings, in both preclinical animal models and on human liver specimens, converge in supporting the presence of specific aging hallmarks in the diseases involving these hepatic compartments.

In some embodiments of the invention, an effective amount of the phospholipid-bound EPA/DHA compounds, or the phospholipid compositions described above, are administered to a subject in need thereof
to treat, prevent, or improve senescence related to age-related pathologies selected from the group of atherosclerosis, cardiovascular dysfunction (including cardiovascular problems caused by certain genotoxic chemotherapies), tumor progression, loss of hematopoietic and skeletal muscle stem cell functions, non-alcoholic fatty liver disease, pulmonary fibrosis, osteoarthritis and osteoporosis

In one a particular preferred embodiment, the composition for use in therapy or in the methods of treatment to prevent or improve senescence related to age-related pathologies as described comprises a phospholipid-bound EPA and/or DHA wherein a predominant amount of EPA and/or DHA is bound to a PC-compound compared to the amount of EPA and/or DHA bound to a LPC.

It has previously been demonstrated that the role of mitochondria is important in the retina, which is one of the body's most bioenergetic organs.⁴⁸ The eye is exposed to visible light and has extensive antioxidant protective mechanisms. Aging retinal pigment epithelial cells have impaired mitochondrial function with increased reactive oxygen species production. It is demonstrated that aging-related mitochondrial dysfunction causes increased oxidative injury, which, coupled with impaired repair mechanisms, results in retinal dysfunction and retinal cell loss, leading to visual impairment. This has been linked to age-related blindness in such as age-related macular degeneration. It has also been 9 implied that mitochondrial dysfunction has a role in diabetic retinopathy and glaucoma.

The transporter Mfsd2, which is involved in LPC-mediated uptake of EPA and DHA, has a wide tissue distribution and is expressed in various organs, such as the brain, retina, liver, kidney, intestinal mucosa, lungs, mammary gland, ovaries, uterus, prostate and testis.

In another particular preferred embodiment, the composition for use in therapy or methods for ameliorating at least one hallmark of aging in a tissue chosen from the group consisting of the brain, retina, liver, kidney, intestinal mucosa, lungs, mammary gland, ovaries, uterus, prostate and testis, comprises a phospholipid-bound EPA and/or DHA wherein a predominant amount of EPA and/or DHA is bound to a LPC-compound compared to the amount of EPA and/or DHA bound to a PC

### EXAMPLES

### Example 1

The purpose of this study was to determine if marine derived phospholipid compositions can have effects on hallmarks of aging. A krill derived phospholipid composition rich in phosphatidylcholine (Superba Boost) was chosen as model substance for this study. The composition of Superba Boost is provided in Table 1.

### Materials and Methods

C. elegans Strains and Culture Conditions

Animals were cultured on nematode growth medium (NGM) plates with the Escherichia coli strain OP50 at 20° C. using standard procedure (Brenner, 1974). The adult animals were bleached to obtain synchronized populations. For all aging associated studies, L4 hermaphrodites were grown in NGM plates containing krill oil (0,5p1/ml) from day 1 to day 6. The aging population of worms were maintained in NGM plates without FdUrd. Instead, animals were washed with M9 buffer and filtered through Nylon Net Filter (catalog #NY4104700) everyday post adult day 2 stage, till the desired age. The adult Day 1 was defined as 24 hours post L4 stage. The following nematode strains were used in this study: N2: wild type; BY273 Is[p_{dat-1}GFP; p_{dat-1} α-syn] to monitor dopaminergic neurons. HLN107 strain was built by crossing TU3401: sid-1 (pk3321) V;uls69 V with BY273 to perform RNAi in dopaminergic neurons. P_{unc-54} α-syn::GFP was used to investigate α-synuclein aggregation. To study Alzheimer disease (AD) using C. elegans; strain BR5270 [P_{rab-3}::F3(delta) K280] and strain CK12 [aex-3::tau4R1N(P301L)+myo-2p::gfp] was used for Tau model; strain CL2355[P_{sub-1}::Aβ₁₋₄₂] was used for Aβ model; strain UM0001[P_{sub-1}::Aβ₁₋₄₂; P_{rab-3}::F3(delta) K280] was used for Tau;Aβ model. In the following, the N2 and BY273 strains will be referred as wildtype (WT) and PD animals respectively. The strains BR5270, CK12, CL2355 and UM0001 will be referred to as AD animals.

### Cell Lines

The human fibroblastoid cell line BJ was grown in Dulbecco's Modified Eagle Medium, GlutaMAX (DMEM, Life Technologies) supplemented with 10% (vol/vol) fetal bovine serum (FBS, Lonza) and 1× Penicillin-Streptomycin (Life Technologies). Cells were grown at 37° C. with 5% CO2.

RNA Isolation and qPCR

Total RNA was isolated with RNeasy mini kit (Qiagen) following the manufacturer's instructions. Reverse transcription was performed using High-Capacity cDNA Reverse Transcription kit (Applied Biosystems). Quantitative PCR was carried out on a QuantStudio 7 Flex detection system (Applied Biosystems) with the Power SYBR green PCR master mix (Applied Biosystems). Each sample was analysed in triplicate.

### Chemicals and Antibodies

The senescence marker X-gal were purchased from Sigma (B4252-250MG Oslo,Norway). Serotonin hydrochloride was from Sigma (Oslo, Norway). The following commercially available antibodies were used: monoclonal 8-oxo-dG (4354-MC-050 Trevigen). Secondary antibodies were Alexa Fluor 555-conjugated anti-rabbit and anti-mouse (Invitrogen). For RNA isolation, Direct-zol RNA Miniprep Kits (#R2050) was used from zymo-research. Senescence marker for fibroblasts, SPiDER-β-gal, was purchased from Dojindo (SG02-10). TMRE, (Mitochondrial membrane potential assay kit) was purchased from Abcam (ab113852). Cell nucleus staining dye Hoechst was purchased from Thermo Scientific (Hoechst 33342).

### Degeneration of Dopaminergic Neurons

The degeneration of dopamine neurons in BY273 Is[p_{dat-1}GFP; p_{dat-1} α-syn]was monitored by following GFP expression under the dopamine transporter (DAT-1) promoter^{16,17}. The L4 larval stage of BY273 animals were exposed to vehicle and krill oil (0,5 μl/ml media). The in vivo imaging of dopaminergic neurons was performed on day 1, day 3 and day 6 old animals. At this stage, 15-20 worms were immobilized on an agar padded glass slide with 2 mM levamisole and glass cover slip. The dopaminergic (DA) neurons were imaged under a Zeiss LSM780 confocal microscope with x20 objective.

### Immunohistochemistry

For immunostaining, adult worms were washed twice with milliQ water. Washed worms were placed on poly-L-lysin-coated slides (Thermo Scientific), and freeze cracked using coverslips on dry an ice block. The primary 8-oxo-dG antibody was used at 1:200 dilution. The secondary antibody, Alexa Fluor 555-conjugated anti-mouse was used at 1:1500 dilution. Prolong gold with DAPI was used for mounting (Invitrogen, P36931). The slides were imaged under Zeiss LSM780 confocal microscope with x63 plan-Apochromat 1.4 NA objective.

### Basal Slowing Response

The basal slowing response was performed as described¹⁶. Well-fed synchronized old day 6 worms were tested. The NGM plates with and without OP50 were used to count body bends per 20 seconds. The locomotion rate was counted after 5 minutes of transfer, to avoid overstimulation. The assay was performed blindfolded, with three independent replicates.

### Pharyngeal Pumping

We used a microfluidics based ScreenChip system from Invivo Biosystem, to detect pharyngeal pumping rate in each individual worm¹⁶. For this assay worms were age synchronized. The worms were first washed twice with M9 buffer, before incubating them with 10 mM serotonin prepared with M9 buffer at room temperature for 30 minutes. Simultaneously, the screenchip fluidics system was prepared by following the user guide. The worms were loaded in 1.5 ml eppendorf tubes with 1 ml M9 buffer and vacuum sucked into the screen-chip using a vacuum pump. Pumping frequency was measured by EPG recording using screen-chip 40 for old day 3 worms. Each recording was 1 to 2 minutes long, with 15-20 worms per genotype.

### Chemotaxis Associated Learning

The short term associated memory training (STAM) assay was performed as described in^{16,18}. The assay is based on the ability of C. elegans to learn and remember a positive association between food and the weak chemoattractant butanone. Briefly, age synchronized worms maintained at 20° C. were divided into naïve and trained groups. Each group had 200 to 300 worms per condition. The trained group was washed three times with M9 and starved for one hour in M9 before training. After two hours of conditioning, the worms were trained to positively associate food and butanone, they were kept at hold for 120 minutes in seeded NGM plates. STAM was measured after two hours intervals of (spaced) training. Worms were tested for chemotaxis toward 10% butanone before (naïve) or after two hours conditioning (Trained). The chemotaxis index was calculated as follows: (CI)=(n_{butanone}−nₑₜₕₐₙₒₗ)/(Total−n_{origin}). Learning Index was calculated by subtracting the naïve CI from the post-conditioning training CI (CI_{butanone}−CI_{naïve}). The memory assay in AD animals were performed as described elsewhere¹⁹.

### Oxygen Consumption Rate

The oxygen consumption rate was measured using DW1/AD clark-type polarographic oxygen sensor (Hansatech Instruments, model: Oxygraph Plus System). The protocol was exactly implemented as described without modification²¹.

### Senescence

The senescence assay was performed as described²¹ with minor modifications. Briefly, the animals were washed with PBS and fixed with 0.2% glutaraldehyde+2% PFA in 1×PBS on dry ice for 5 minutes. Next, the animals were thawed in room temperature for 10 minutes and stained with freshly prepared X-gal staining solution containing lmg/ml X-gal (stock 20 mg/ml in DMSO), 40 mM citric acid/sodium phosphate pH 6.5 mM potassium ferrocyanide, 5 mM potassium ferricynide, 150 mM NaCl and 2 mM MgCl2 and incubated overnight at 37° C. covered in dark. The stained animals were washed with 1×PBS and mounted on glass slides with coverslips. The animals were imaged under a bright field microscope with x20 objective.

Senescence assay in BJ fibroblasts was done using Spider-β-Gal marker kit purchased from Dojindo. 50,000 (late passaged) fibroblasts cells were seeded on 35 mm imaging dish (from IBIDI) with polymer coverslip bottom and cells were allowed to grow overnight at 37° C. in 5% CO2 incubator. 24 hours post seeding, cells were treated with 100 μg/mL Krill Oil and allowed to grow for 6 days at 37° C. in 5% CO2 incubator. Senescence assay on live cells was performed on day 7 according to the manufacturer protocol. Briefly, cells were washed once with PBS and then treated with 1 mL of Bafilomycin working solution and incubated at 37° C. for 1 hour in a 5% CO2 incubator. After 1 hour, 1 mL of SPiDER—β-Gal working solution containing 2 μg/mL Hoechst was added to the cells and they were incubated again for 30 minutes in a 5% CO2 incubator. After incubation, cells were washed with PBS and then imaged under Zeiss LSM780 confocal microscope at 63× with appropriate filters.

### TMRE Staining

A TMRE (Tetra methyl rhodamine, ethyl ester) Mitochondrial Membrane Potential assay kit (cat. no. ab113852; Abcam) was used to measure the mitochondrial membrane potential. In total, (late passaged) 50,000 BJ fibroblasts cells were plated onto 35 mm imaging dish (from IBIDI) with polymer coverslip bottom. 24 hours post seeding cells were treated with 100 μg/mL Krill Oil and the cells were allowed to grow for another 5 days at 37° C. in 5% CO2 incubator. On day 6, TMRE assay was performed according to the manufacturer protocol. Briefly, cells were stained at a final concentration of 200 nM TMRE and incubated in dark for 30 min at 37° C. After incubation, cells were washed again with PBS and live cell imaging was done on Zeiss LSM780 confocal microscope, at 63× magnification using appropriate filters.

### Mitochondria Copy Number and Gene Expression Analysis

Mitochondrial DNA (mtDNA) copy number was quantified using droplet digital PCR (ddPCR). Briefly, the age synchronized wildtype and PD animals were individually picked in lysis buffer (TE low containing 1 mg/ml proteinase K and 0.01 mg/ml RNase). The lysis was performed for 1 hr in 65° C. followed by 95° C. for 15 minutes. From here this method was performed exactly as described in¹⁶.

For Mitochondrial gene expression analysis, transcriptional activation of ctc-1 and nduo-2 was measured in age synchronized wildtype and PD animals. The Single worms were collected in 1 ml PBS and immediately snap frozen in dry ice. The protocol was exactly implemented as described without modification¹⁶.

### RNA Isolation for RNAseq

Age synchronized day1, day3, day 6 wildtype and PD animals were treated, or not, with krill oil. These animals were harvested and RNA isolation was performed the same day. Briefly, the animals were washed x2 with sterilized milliQ water, and collected in 1.5 ml Eppendorf. The worm pellets were dispensed in 600 μl of TRI reagent or trizol and transferred to tube with sterile beads to homogenize the animals. From here RNA isolation for tissue was followed as described in the Direct-zol RNA Miniprep Kits protocol section. The isolated RNA was dissolved in 10 μl nuclease free water and the quality was analyzed in bioanalyser.

### RNA Sequencing

Sequencing libraries were prepared from 200 ng total RNA using Nugen Universal Plus Total RNA-Seq library preparation kit (Tecan) with custom AnyDeplete design for C. elegans. Final libraries were pooled (10 samples/run) and paired-end sequencing (2×71 bp) performed using NextSeq High Output kits on the NextSeq 550 sequencer. The raw sequencing data were demultiplexed using BCL Convert (Illumina) and the sequencing reads were aligned to the C. elegans WBcel235 cDNA with kallisto²² and UMI-duplicates removed with umi-tools²³. Depulicated bam-files were converted to fastq with samtools²⁴ before quantifying abundances of transcripts with kallisto.

### RNA Seq Data Analyses

All analyses were done using R version 4.1.0 on a x86_64-pc-linux-gnu (64-bit) platform, running the Pop!_OS operating system, version 21.04. The computer used for the analyses has 32 Gb of RAM with 4 8th generation i5 cores, each threaded twice resulting in 8 available CPUs. Differential gene expression was performed using the DESeq2 R package²⁵, version 1.32.0. Count files were split into their respective groups and attributed defining conditions, such as ‘control’ and ‘experiment’. The counts for the respective conditions are merged into one dataframe, from this dataframe, genes which have no counts for every sample are removed. A sample defining file containing the sample names along with their associated condition is also created, this file contains the relevant information for the samples within the merged count file. The values are then normalized using the DESeq2 normalization method. The standard DESeq2 pipeline was used to obtain the differential gene expression results. It starts by creating the DESeqDataSet-class by calling the DESeqDataSetFromMatrix function, using the merged count file, and the sample defining file are used as inputs, while the condition is used as the design. The design always uses the ‘experiment’ and compares it with the ‘control’. Using the estimateSizeFactors function, the median ratio method is utilized to estimate the size factors of the DESeqDataSet object. The DESeq2 function is called, which performs an estimate of dispersion followed by a negative binomial GLM fitting and wal statistics in order to obtain the differential gene expression results. Selection of significant differentially expressed genes was made based on an adjusted p value (false discovery rate) below 0.05 and a log 2-fold change value greater than 1 or smaller than −1.

Gene set enrichment analysis (GSEA) was performed using the ClusterProfiler package²⁶, version 4.0.0. The analysis is run for the biological process (BP) ontology. The AnnotationDbi package, version 1.54.1, was used to load the information relating to the studied organisms. Using this package, the background of the GSEA was set using the org.Hs.eg.db library, version 3.13.0. The GSEA was performed on differential gene expression analysis, where every gene within the differential expression results, regardless of significance, is inputted for the analysis. The pvalue and false discovery rate (FDR) cutoffs are set to 0.05. The semantic distance is added to the GSEA results using the pairwise_termsim function from the enrichplot package, version 1.12.1. The similarity matrix added is calculated using the ‘Wang’ method²⁷.

The WGCNA package²⁸, version 1.70-3 was used to obtain a weighted correlation association between gene modules and behavioral parameters. WGCNA takes in RNAseq count data. A sample file was manually prepared for the WGCNA package, the sample file details which behavioral values are associated with the samples inputted. The pipeline first removes any samples with too many missing values in proportion with the amount of samples and values inputted. The samples are then clustered and their power is evaluated. Based on the indications of²⁸, the power value used is the first value above the 0.8 threshold which was a value of 4. The blockwise Modules function is used to calculate the gene modules and obtains the correlation values with the clinical parameters. Each gene module is run through gprofiler²⁹ in order to extract potential biological relevance of each gene module.

Biological age predictions were performed using the fastq files as raw data as input in the preprocessing specified in the Bit age pipeline. First, the data were processed with fastp version 0.23.2 with parameters −g −x −q 30 −e 30 −w 8. And second, reads were aligned with STAR aligner and parameters --quantMode GeneCounts --runThreadN 8, --outSAMtype BAM Unsorted --sjdbOverhang 69 and again the ones suggested by STAR manual in ENCODE options, as above. For predicting biological age we took raw counts from the STAR aligner output and computed count per million(CPM), as required by bit age method, with edgeR library version 3.38.1. We then computed predicted biological age using elastic net coefficients and code provided in the bit age material

### Results

Krill Oil Protects Dopaminergic Neurons from Age-Related Degeneration Improves Dopamine-Dependent Behavior

To determine whether growth in the presence of krill oil protected DA neurons from degenerating over time, we used the C. elegans Parkinson's disease (PD) model, a humanized strain where overexpression of α-synuclein under control of the dat-1 promoter exacerbates age-dependent degeneration of dopaminergic (DA) neurons^{12,16}. As previously reported, we saw substantial reduction of the number of DA neurons in day 6 old animals compared to the younger adults (day 1 and day 3) both when assessed by fluorescence intensity of the DA neuronal GFP (FIG. 1a) and by scoring the number of DA neurons by differential interference contrast (DIC) microscopy (FIG. 1b). Growth in media enriched with krill oil resulted in a remarkable protection of DA neurons from age-related degeneration (FIG. 1a and 1 b). As expected, aging reduced the fraction of DA neurons to 60% in day 6 adults compared to 90% in day 1 adults when grown on standard media. In contrast, in krill oil-treated animals there was no reduction of the fraction of surviving DA neurons at day 3 to day 6 (FIGS. 1a and 1b). Moreover, there was a small increase in the fraction of surviving DA neurons in day 1 animals, suggesting a general protection against α-synuclein mediated proteotoxicity.

To test whether krill oil suppressed α-synuclein aggregate formation, a key pathogenic hallmark of Parkinson's disease, we used a reporter strain expressing α-synuclein coupled with GFP in body wall muscle cells¹⁶. The day 1 young animals showed no evidence of α-synuclein aggregation, whereas the day 6 old animals had clear presence of α-synuclein aggregates, averaging about 50 aggregates per animal. Interestingly, krill oil treated animals showed a significant reduction of α-synuclein aggregation, with an average of about 20 aggregates (FIG. 1c and 1d).

In the C. elegans PD model, dopamine-regulated behaviors are well characterized³⁰. Many groups, including us, have demonstrated that DA neuron-dependent behavior like basal slowing response (BSR) and movement ability deteriorates with age¹⁶. Therefore, we wanted to determine whether the improvement of DA neuron survival led to preservation of DA neuron function, we examined BSR in both wildtype and day 6 old PD animals. Intriguingly, krill oil-fed animals had significantly higher BSR than untreated animals (FIG. 1e). Further supporting improved DA—dependent behavior performance, day 6 old animals showed an average activity score of 400/hour as compared to only 340/hour in untreated PD animals. Whereas the wildtype animals showed no change in response to krill oil (FIG. 1f). Clearly suggesting that, old PD animals fed krill oil showed improved movement activity as compared to untreated animals. These findings suggest that krill oil protects DA neurons from degeneration, inhibits α-synuclein aggregate formation, and improve dopamine dependent behaviors.

Krill Oil Reduces Senescence in C. elegans and Human Fibroblasts

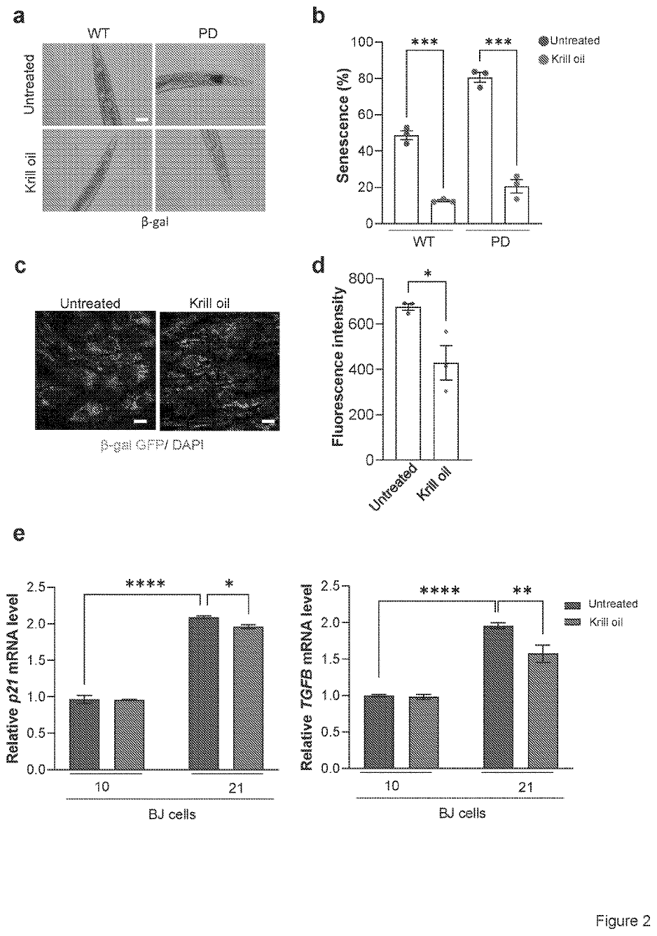

Senescence is a hallmark of aging and evidence suggests that senescence-like atrophy may be responsible for age-dependent loss of germline gonadal cells and self-destruction of intestinal biomass in C. elegans³¹⁻³³. In 9-day old animals, 45% of wildtype animals and 80% of PD animals exhibited positive β-gal staining. When these animals were fed krill oil, wildtype and PD animals showed 35% and 60% reduction of positive β-gal staining, respectively (FIGS. 2a and 2b).

Since it is unclear whether C. elegans experiences a process similar to the senescence-associated secretory phenotype, which is an effector of senescence-driven aging in human tissue, we used human BJ fibroblast and treated these cells with krill oil to support our data from worms. Late passage BJ fibroblasts, which are widely accepted as a model to follow senescence, exhibited 1.75-fold reduction in β-gal positive cells (FIGS. 2c and 2d) as compared to the untreated cells. The reduction in senescence in late passage BJ cells were accompanied by reduced expression of the senescence markers p21 and TGFβ (FIG. 2e). Altogether, these data show that krill oil suppresses senescence in both C. elegans and in human fibroblasts.

### Krill Oil Improves Mitochondrial Health

Oxidative stress is a fundamental feature of aging and a pathogenic factor in Parkinson's disease^{1,16} Reactive oxygen species (ROS) generated due to metabolic activity mitochondria are an important source of oxidative stress in brain cells. ROS cause oxidative DNA damage in both nuclear and mitochondrial DNA³⁴. Oxidation of guanine, 8-oxoG, is a biomarker of nucleic acid oxidation and, as previously demonstrated¹⁶, PD animals accumulate 8-oxoG with age (FIGS. 3a and 3b). Because DHA and astaxanthin are components of krill oil with antioxidant properties, we were interested to see how 8-oxoG levels were affected. Notably, compared to untreated animals, krill oil supplemented day 6 old PD animals showed a remarkable, 6-fold decrease in 8-oxoG levels, suggesting attenuation of oxidative stress.

As mitochondrial dysfunction is a main source of oxidative stress, we tested whether krill oil may prevent age-related decrease of mitochondrial function by monitoring the oxygen consumption rate (OCR). OCR declines with aging²⁰. Krill oil suppressed age-related reduction in OCR in PD animals where a drop in OCR was only apparent in day 6 old animals compared to a clear drop in OCR in the untreated animals as early as day 3 (FIG. 3c). This was not attributable to a rise in mitochondrial copy number which stayed unchanged in both wildtype and PD old day 6 animals in response to krill oil (Data not shown). Clearly indicating that krill oil promotes mitochondrial health in PD animals.

To test whether krill oil also promoted mitochondrial health in human cells, we measured the mitochondrial membrane potential using tetramethylrhodamine ethyl ester (TMRE), a dye that is imported into mitochondria as a function of the mitochondrial membrane potential and, thus, specifically stains healthy mitochondria. The fluorescence intensity of TMRE was substantially reduced in late passage human BJ fibroblasts. Notably, after only 5 days of treatment with krill oil, these cells showed a remarkable 1,2-fold increase in TMRE fluorescence intensity (FIG. 3 d-e). Taken together, these data strongly indicate that krill oil enhances mitochondrial health in both C. elegans and human fibroblasts.

Krill Oil Modulates Gene Expression Profile with Age

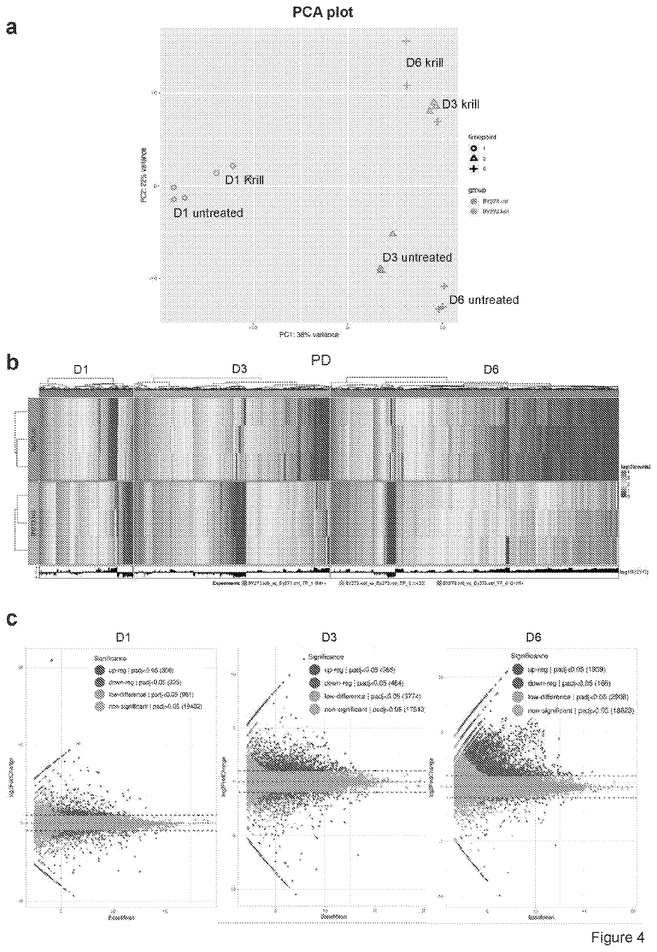

In the above we showed that krill oil has widespread impact on many phenotypic hallmarks of aging. To get an unbiased view of the pathways activated by krill oil in a life-course perspective we used RNA sequencing. Data was harvested from day 1, 3, and 6 old wildtype and PD animals. The principal component analysis (PCA) plot showed good separation of experimental groups with respect to age (from day 1, day 3 and day 6) and treatment (FIG. 4a; supplementary data not shown). In PD animals the variation between treatment groups in day 1 animals is quite low as compared to 22% variance in day 3 and day 6 animals (FIG. 4a). The technical quality of the RNAseq analysis was confirmed in a PCA plot by representing the clustering of the three biological replicates with and without krill oil treatment in both wildtype and PD animals. For the wildtype strain the PC2 variance ranges from 17, 10 and 8% in between each biological replicates in day 1, 3 and 6 respectively. Whereas the condition variation PC1, is 57, 73 and 81% in day 1, day3 and day 6 respectively (data not shown). Similarly, in the PD animals, the PC2 varies from 14, 5 and 18% in between each biological replicates in day 1, 3 and 6 respectively. The condition variation is quite large, as expected, which ranges from 64, 87 and 71% in day 1,3 and 6 animals respectively (data not shown).

Differentially gene expression analyses showed that with age there is an increase in the number of differentially expressed genes (DEGs) in day 1, day 3 and day 6 animals treated with krill oil. Volcano plots illustrate that the number of upregulated DEGs increased with age from 356 genes at day 1, 965 at day 3, to 1939 at day 6. Interestingly, day 6 old PD animals showed higher number of upregulated DEG as compared to age matched wildtype population (FIG. 4b). Whereas maximum number of downregulated genes were found at day 3 (FIG. 4c). Thus, krill oil affects gene expression more in older animals than in young animals. In summary, it was found that krill oil induces temporal transcriptome rewiring resulting in reduced biological age estimates, thus promoting healthy aging. The biological age estimates were calculated using the BiT age calculator (Meyer and Schumacher. Aging Cell 20, e13320 https://doiorg.ezproxy.uio.no/10.1111/acel.13320 (2021).).

### Cluster Analysis

To identify groups of genes that respond similarly to krill oil, we used gene cluster analysis. DEGs with an absolute log 2 fold change greater than one were included in the analysis. Several clusters containing genes which temporally was co-regulated in response to krill oil were identified (FIG. 5a,supplementary data not shown). For example, the clusters 4, 6, and 7 comprising 231, 47 and 61 genes, respectively, were upregulated in the animals treated with krill oil from day 1 to day 6. Gene ontologies (GOs) enriched in C4 were associated to neuroprojection morphogenesis; C6 with neurogenesis, cuticle structure, metalloendopiptidase activity; and C7 with regulation of cell differentiation, cuticle development, structural constituent of collagen (FIG. 5a, supplementary data not shown). The upregulation of genes involved in neurogenesis and neuron projections in the PD animals treated with krill oil were highly interesting in light of the increased survival of dopaminergic neurons (FIGS. 1a and b). Moreover, genes involved in cuticle development were accompanied at the phenotypic level with intact and tight cuticle in 9-day old PD animals fed krill oil compared to the wrinkled cuticle in untreated animals as shown by transmission electron microscopy (TEM) (data not shown). Several clusters, e.g. C5, C8, C9, C10 and C12 comprised genes that were highly upregulated on day 3 but dropped at day 6 in krill oil treated PD animals (FIG. 5a, supplementary data not shown), although the expression in day 6 is significantly higher than day 1. The cluster C8 has the highest number of gene (527), comprising GOs involved in structure of cuticle, cytoskeletal protein binding activity, neuron projection, presynaptic membrane, axon and synapse, MAP kinase- and calcium signaling pathways, neurotransmission of cholinergic neurons (FIG. 5b). Again, these GOs are consistent with the protection of DA neurons and intact cuticle in PD animals fed with krill oil (FIGS. 1a and 1b; supplementary data not shown). It is well recognized that MAP kinases modulate oxidative stress regulators³⁵′3⁶ and senescence, which corresponds to the enriched GOs in cluster 8 (FIGS. 5a and 5b). C9 comprises 194 genes involved in cell-cell, gap-junction, cytoskeletal protein binding (data not shown). The enriched GOs in cluster C10 include 116 genes involved in male sex determination, genitalia development, reproductive system development, and glycerophospholipid metabolism (data not shown). Cluster 12 represents 122 upregulated genes which are annotated to the GOs cell signaling, neuron projection, synapse organization and oral pharyngeal dysphagia (FIGS. 5a and 5c). Interestingly, genes in this cluster were downregulated in the untreated PD animals, indicating that krill oil may counteract age-related changes in gene expression.

Genes downregulated in response to krill oils are found in clusters C16, C22, C30 and C31 (data not shown). These clusters comprise 218, 193, 41 and 73 genes respectively. The cluster C16 encompasses genes regulating cell development, oxidoreductase activity, heme binding proteins, iron-ion binding proteins. In early development, UDP-glucuronosyltransferase is highly upregulated and governs hormonal signaling³⁷. In krill oil-fed PD animals, the expression of UDP-glucuronosyltransferase is reduced in clusters 22 and 31. Clusters C 30 and C 31 predicts that krill oil suppresses several biosynthetic processes, e.g. pantothenate biosynthesis, through development and adult stages.

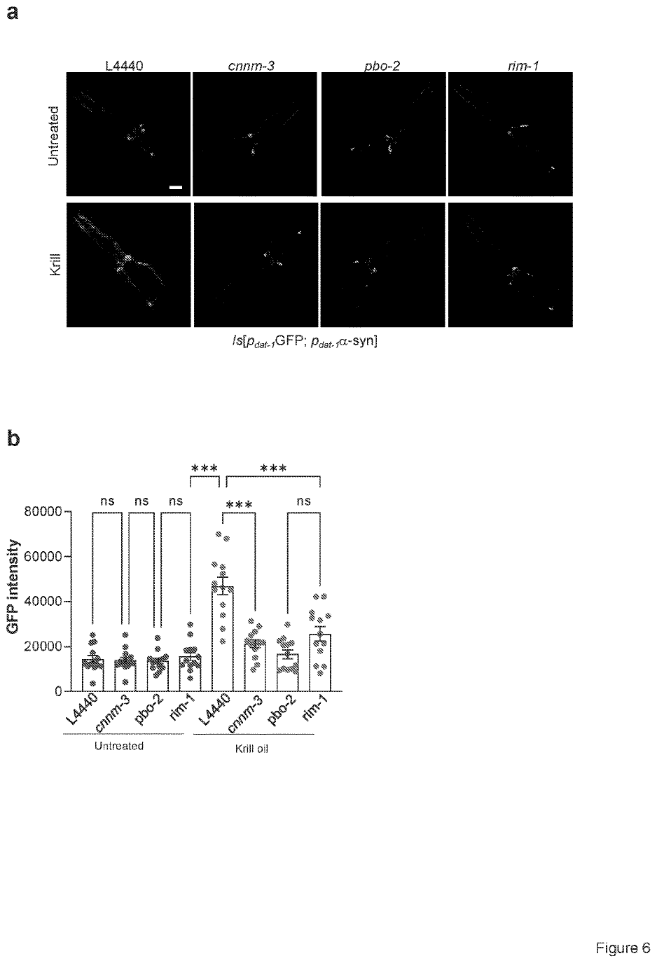

To test whether these changes in gene expression is indeed attenuating DA neuron degeneration in old animals, we scored the dopaminergic neuron survival after RNAi mediated knock-down (FIGS. 6a and 6b). Upon depletion of cnnm-3, pbo-2 and rim-1 (human homologs of CNNM3, PLCB4 and RIMS1 respectively), the neuroprotective effect of krill oil was abolished in the PD strain, while no effect was seen in the wild type background. Thus, this supports that krill oil induces global changes in gene expression in several pathways that promotes DA neuron survival (FIGS. 6a and 6b).

### WGCNA Clustering

To further define which components of the global transcription reprogramming that correlated with changes in phenotypic end-points, we used WGCNA analysis. This approach first clusters the genes into ‘modules’, each module is color coded. Thus, the different colors represent groups of genes which have similar trajectories using WGCNAs clustering method. Then the modules were correlated with defined clinical parameters. We used the three endpoints; Survival fraction of DA neurons; oxygen consumption rate and locomotion ability in all the time points (day1, day 3 and day 6). We also added krill oil treatment as a parameter to check if it correlates with the presence of krill oil or not. The pipeline gives an unbiased analysis and treats every input with equal weight, either control or treated PD animals in all three time points, and find correlation between genes and clinical parameters, but not necessarily a biological event such as the presence or absence of krill oil.

Correlation values between all of the modules identified and defined parameters is shown as a heatmap (FIG. 7a). The correlation ranges from +1 (completely positively correlated) to −1 (perfect negative correlation). Where a positive correlation indicates that an increase in the gene expression results in an increase of the value of that parameter. Negative correlation indicates an inverse reaction.

The green module has a strong negative correlation with the survival of the DA neurons (FIGS. 7a and 7b), this indicates that the genes in this group are less expressed when DA neurons have high survival and they are downregulated in the presence of krill oil. Similarly, the blue module has a strong positive correlation with krill oil animals and motility (FIG. 7 a-c), this indicates that the genes in this group are highly expressed, and correlated with intact motility performance in the presence of krill oil. The gprofiler shows the GOs that are associated to the green and blue modules (FIGS. 7b and 7c). The skyblue module shows a strong correlation with neuron survival, however many of these are not functionally characterized and gprofiler gives only three hits which does not predict any associated pathways. The trajectories show that genes in the green module spike at day 3 in the control as opposed to a very stable expression in krill (FIG. 7b). For genes in the blue module, trajectories are defined by dramatical increases the expression in old animal (FIG. 7c). Altogether, we conclude that krill oil modulated separate gene expression programs in mid-life and in old animals. Interestingly, changes in gene expression programs in mid-life adults also promoted healthy aging by attenuating several hallmarks of aging, resulting in remarkable protection of DA neuron survival in aging animals.

### Validation in Human BJ Cell Line

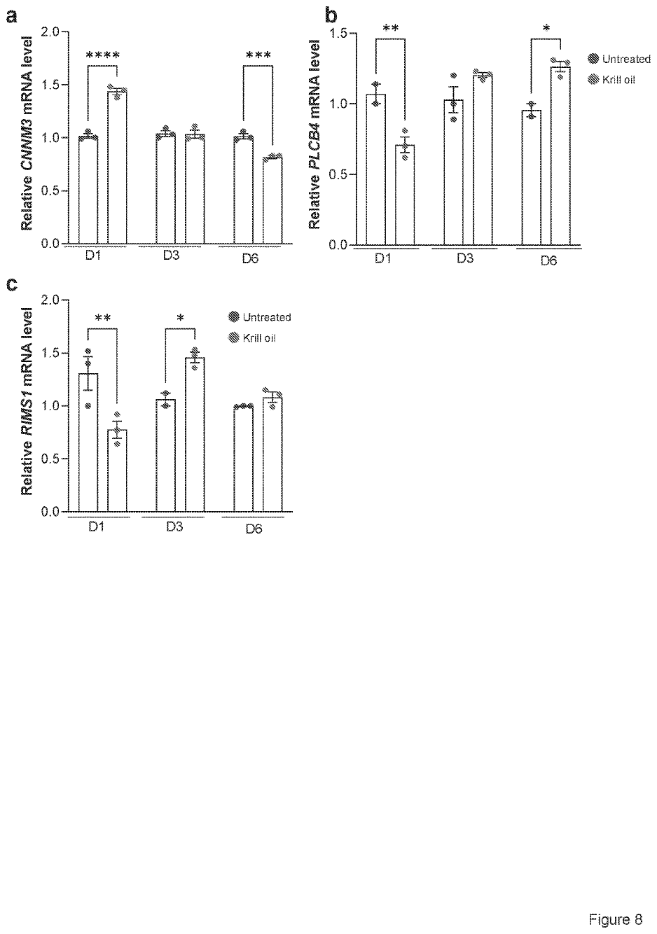

To test whether features of the gene expression changes were conserved in human cells, we measured temporal changes in expression of human orthologs of selected, upregulated genes from C12 (BRSK1 and KIRREL1), C4 (PRPS1) and C8 (CNNM3, PDE3A, PLCB4, RIMS1) in krill oil treated human fibroblasts. First human BJ cell lines were cultured for 10 passages prior to krill oil treatment. Gene expression was measured by q-RT PCR after 1, 3, and 6 days of treatment; CNNM3 and PLCB4 mRNA expression was significantly increased in krill oil treated BJ fibroblast on day 1 and day 6 respectively (FIGS. 8a and 8b). RIMS1 mRNA, encoding the RIMS1 protein that regulates neurotransmitter release upregulated in BJ cells after 3 days exposure to krill oil but not after 6 days, a temporal regulation that was also seen for the C. elegans ortholog in C8, with upregulation in mid-life, but not in the old animals (FIG. 8c and FIG. 5a). Thus, although cell cultures cannot capture aging per se, the dynamic changes in gene expression seen in C. elegans was largely reciprocated also in human cells.

### Oxidative Stress Regulator Dependent Protection of Dopaminergic Neurons in Response to Krill Oil

As Krill oil contain compounds with antioxidant and anti-inflammatory properties, we investigated whether oxidative stress regulators, which are frequently implicated in promoting healthy aging 16, was required for DA neuron protection. Again, RNAi was used to downregulate the central oxidative stress regulators JNK-1, SKN-1, and LMD-3. Wild type animals showed no obvious difference in DA neuron survival at day 6 after down regulating the jnk-1, skn-1, and lmd-3 genes. Conversely, the protection of DA neurons was lost in PD animals after depleting skn-1 and lmd-3. However, jnk-1 knock down had no effect (FIGS. 9a and 9b). Clearly indicating that DA neuroprotection mediated by Krill oil is dependent on core oxidative stress regulators important for regulating pathways that promote healthy aging. Intact dopaminergic neurons regulate pharyngeal pumping 31.

We observed that krill oil fed animals displayed improved pharyngeal pumping in PD animals, which is a commonly accepted health span metric in C. elegans (FIG. 9c). Moreover, dopamine deficiency impairs chemosensory learning by affecting olfactory response 39. Consistently, Krill oil-fed PD animals showed significant improvement of chemotaxis-associated learning capacity (FIG. 9d). However, the wildtype animals showed no significant benefit for pharyngeal pumping or cognition improvement in response to krill oil (FIGS. 9c and 9d). Altogether, we can conclude that krill oil does improves dopamine dependent behaviors and cognition in C. elegans.

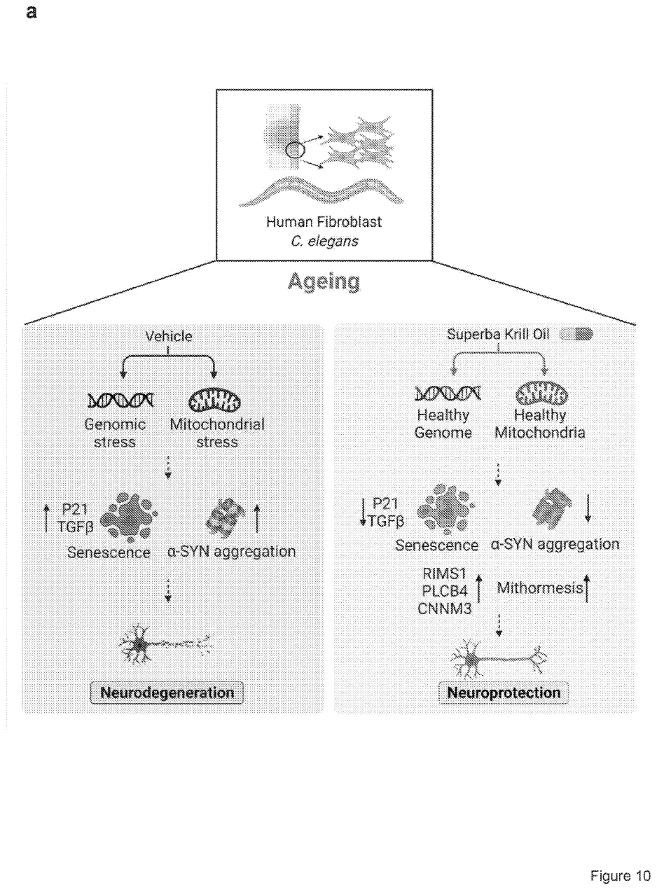

FIG. 10 provides a schematic depiction of the effect of krill oil on aging hallmarks in C. elegans.

### REFERENCES

1 López-Otín, C., Blasco, M. A., Partridge, L., Serrano, M. & Kroemer, G. The hallmarks of aging. Cell 153, 1194-1217, doi:10.1016/j.cell.2013.05.039 (2013).
2 Aman, Y. et al. Autophagy in healthy aging and disease. Nat Aging 1, 634-650, doi:10.1038/s43587-021-00098-4 (2021).
3 Hansen, M., Rubinsztein, D. C. & Walker, D. W. Autophagy as a promoter of longevity: insights from model organisms. Nat Rev Mol Cell Biol 19, 579-593, doi:10.1038/s41580-018-0033-y (2018).
4 Ierna, M., Kerr, A., Scales, H., Berge, K. & Griinari, M. Supplementation of diet with krill oil protects against experimental rheumatoid arthritis. BMC Musculoskelet Disord 11, 136, doi:10.1186/1471-2474-11-136 (2010).
5 Ambati, R. R., Phang, S. M., Ravi, S. & Aswathanarayana, R. G. Astaxanthin: sources, extraction, stability, biological activities and its commercial applications—a review. March Drugs 12, 128-152, doi:10.3390/md12010128 (2014).
6 Yalagala, P. C. R., Sugasini, D., Zaldua, S. B., Tai, L. M. & Subbaiah, P. V. Lipase Treatment of Dietary Krill Oil, but Not Fish Oil, Enables Enrichment of Brain Eicosapentaenoic Acid and Docosahexaenoic Acid. Mol Nutr Food Res 64, e2000059, doi: 10. 1002/mnfr.202000059 (2020).
7 Bengtson Nash, S. M., Schlabach, M. & Nichols, P. D. A nutritional-toxicological assessment of Antarctic krill oil versus fish oil dietary supplements. Nutrients 6, 3382-3402, doi:10.3390/nu6093382 (2014).
8 Choi, J. Y. et al. Antarctic Krill Oil Diet Protects against Lipopolysaccharide-Induced Oxidative Stress, Neuroinflammation and Cognitive Impairment. Int J Mol Sci 18, doi:10.3390/ijms18122554 (2017).
9 Sugasini, D., Thomas, R., Yalagala, P. C. R., Tai, L. M. & Subbaiah, P. V. Dietary docosahexaenoic acid (DHA) as lysophosphatidylcholine, but not as free acid, enriches brain DHA and improves memory in adult mice. Sci Rep 7, 11263, doi:10.1038/s41598-017-11766-0 (2017).
10 Scheinman, S. B. et al. LPC-DHA/EPA-Enriched Diets Increase Brain DHA and Modulate Behavior in Mice That Express Human APOE4. Front Neurosci 15, 690410, doi:10.3389/fnins.2021.690410 (2021).
11 Tissenbaum, H. A. Using C. elegans for aging research. Invertebr Reprod Dev 59, 59-63, doi: 10.1080/07924259.2014.940470 (2015).
12 Lakso, M. et al. Dopaminergic neuronal loss and motor deficits in Caenorhabditis elegans overexpressing human alpha-synuclein. J Neurochem 86, 165-172, doi:10.1046/j.1471-4159.2003.01809.x (2003).
13 Cao, S., Gelwix, C. C., Caldwell, K. A. & Caldwell, G. A. Torsin-mediated protection from cellular stress in the dopaminergic neurons of Caenorhabditis elegans. J Neurosci 25, 3801-3812, doi:10.1523/jneurosci.5157-04.2005 (2005).
14 Kuwahara, T. et al. Familial Parkinson mutant alpha-synuclein causes dopamine neuron dysfunction in transgenic Caenorhabditis elegans. J Biol Chem 281, 334-340, doi: 10.1074/jbc.M504860200 (2006).
Cooper, A. A. et al. Alpha-synuclein blocks ER-Golgi traffic and Rabl rescues neuron loss in Parkinson's models. Science 313, 324-328, doi:10.1126/science.1129462 (2006).
16 SenGupta, T. et al. Base excision repair causes age-dependent accumulation of single-stranded DNA breaks that contribute to Parkinson disease pathology. Cell Rep 36, 109668, doi:10.1016/j.celrep.2021.109668 (2021).
17 Palikaras, K., SenGupta, T., Nilsen, H. & Tavernarakis, N. Assessment of dopaminergic neuron degeneration in a C. elegans model of Parkinson's disease. STAR Protocols 3, 101264 (2022).
18 Kauffman, A. et al. C. elegans positive butanone learning, short-term, and long-term associative memory assays. e2490 (2011).
19 Fang, E. F. et al. Mitophagy inhibits amyloid-β and tau pathology and reverses cognitive deficits in models of Alzheimer's disease. Nat Neurosci 22, 401-412, doi:10.1038/s41593-018-0332-9 (2019).
20 Palikaras, K. & Tavernarakis, N. Measuring Oxygen Consumption Rate in Caenorhabditis elegans. Bio Protoc 6, doi:10.21769/BioProtoc.2049 (2016).
21 Debacq-Chainiaux, F., Erusalimsky, J. D., Campisi, J. & Toussaint, O. Protocols to detect senescence-associated beta-galactosidase (SA-betagal) activity, a biomarker of senescent cells in culture and in vivo. Nat Protoc 4, 1798-1806, doi:10.1038/nprot.2009.191 (2009).
22 Bray, N. L., Pimentel, H., Melsted, P. & Pachter, L. Near-optimal probabilistic RNA-seq quantification. Nat Biotechnol 34, 525-527, doi: 10.1038/nbt.3519 (2016).
23 Smith, T., Heger, A. & Sudbery, I. UMI-tools: modeling sequencing errors in Unique Molecular Identifiers to improve quantification accuracy. Genome Res 27, 491-499, doi:10.1101/gr.209601.116 (2017).
24 Danecek, P. et al. Twelve years of SAMtools and BCFtools. Gigascience 10, doi:10.1093/gigascience/giab008 (2021).
25 Love, M. I., Huber, W. & Anders, S. Moderated estimation of fold change and dispersion for RNA-seq data with DESeq2. Genome Biol 15, 550, doi:10.1186/s13059-014-0550-8 (2014).
26 Yu, G., Wang, L. G., Han, Y. & He, Q. Y. clusterProfiler: an R package for comparing biological themes among gene clusters. Omics 16, 284-287, doi:10.1089/omi.2011.0118 (2012).
27 Zhao, C. & Wang, Z. GOGO: An improved algorithm to measure the semantic similarity between gene ontology terms. Sci Rep 8, 15107, doi:10.1038/s41598-018-33219-y (2018).
28 Langfelder, P. & Horvath, S. WGCNA: an R package for weighted correlation network analysis. BMC Bioinformatics 9, 559, doi:10.1186/1471-2105-9-559 (2008).
29 Raudvere, U. et al. g:Profiler: a web server for functional enrichment analysis and conversions of gene lists (2019 update). Nucleic Acids Res 47, W191-w198, doi: 10.1093/nar/gkz369 (2019).
30 Cooper, J. F. & Van Raamsdonk, J. M. Modeling Parkinson's Disease in C. elegans. J Parkinsons Dis 8, 17-32, doi:10.3233/jpd-171258 (2018).
31 de la Guardia, Y. et al. Run-on of germline apoptosis promotes gonad senescence in C. elegans. Oncotarget 7, 39082 (2016).
32 Ezcurra, M. et al. C. elegans eats its own intestine to make yolk leading to multiple senescent pathologies. Current Biology 28, 2544-2556. e2545 (2018).
33 Kern, C. C. et al. C. elegans feed yolk to their young in a form of primitive lactation. Nature communications 12, 1-11 (2021).
34 McKinnon, P. J. DNA repair deficiency and neurological disease. Nat Rev Neurosci 10, 100-112, doi:10.1038/nrn2559 (2009).
35 Kassahun, H. et al. Constitutive MAP-kinase activation suppresses germline apoptosis in NTH-1 DNA glycosylase deficient C. elegans. DNA Repair (Amst) 61, 46-55, doi:10.1016/j.dnarep.2017.11.009 (2018).
36 Mendelson, K. G., Contois, L. R., Tevosian, S. G., Davis, R. J. & Paulson, K. E. Independent regulation of JNK/p38 mitogen-activated protein kinases by metabolic oxidative stress in the liver. Proc Natl Acad Sci USA 93, 12908-12913, doi:10.1073/pnas.93.23.12908 (1996).
37 Neumann, E. et al. Age-Dependent Hepatic UDP-Glucuronosyltransferase Gene Expression and Activity in Children. Front Pharmacol 7, 437, doi:10.3389/fphar.2016.00437 (2016).
38 Fang, E. F. et al. Tomatidine enhances lifespan and healthspan in C. elegans through mitophagy induction via the SKN-1/Nrf2 pathway. 7, 46208 (2017).
39 Bettinger, J. C. & McIntire, S. L. State-dependency in C. elegans. Genes Brain Behav 3, 266-272, doi:10.1111/j.1601-183X.2004.00080.x (2004).
40 Grimmig, B. et al. Astaxanthin is neuroprotective in an aged mouse model of Parkinson's disease. Oncotarget 9, 10388-10401, doi: 10.18632/oncotarget.23737 (2018).
41 Freitas-Rodrfguez, S., Folgueras, A. R. & López-Otín, C. The role of matrix metalloproteinases in aging: Tissue remodeling and beyond. Biochim Biophys Acta Mol Cell Res 1864, 2015-2025, doi:10.1016/j.bbamcr.2017.05.007 (2017).
42 Tower, J. Sex-Specific Gene Expression and Life Span Regulation. Trends Endocrinol Metab 28, 735-747, doi:10.1016/j.tem.2017.07.002 (2017).
43 Atamna, H. & Frey, W. H., 2nd. A role for heme in Alzheimer's disease: heme binds amyloid beta and has altered metabolism. Proc Natl Acad Sci USA 101, 11153-11158, doi:10.1073/pnas.0404349101 (2004).
44 Alvarez-Cordoba, M. et al. Precision medicine in pantothenate kinase-associated neurodegeneration. Neural Regen Res 14, 1177-1185, doi: 10.4103/1673-5374.251203 (2019).
45 van Deursen JM. The role of senescent cells in ageing. Nature 2014; 509:439 446.
46 Campisi J, Kapahi P, Lithgow G J, Melov S, Newman J C, Verdin E. From discoveries in ageing research to therapeutics for healthy ageing. Nature 2019; 571:183-192
47 Baiocchi et al. Hepatol Commun. 2021 Apr. 10; 5(7):1125-1137. doi: 10.1002/hep4.1725. eCollection 2021 July.
48 Eells J.T. Mitochondrial dysfunction in the aging retina. Biology. 2019; 8:31. doi: 10.3390/biology8020031.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method for ameliorating at least one aging hallmark in a subject in need thereof comprising administering to the subject in need thereof an effective amount of a composition comprising phospholipids having bound eicosapentaenoic acid (EPA) and/or docosahexaenoic acid (DHA), wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication.

2. A method for improving dopaminergic neuron survival in a subject in need thereof comprising administering to the subject in need thereof an effective amount of a composition comprising phospholipids having bound eicosapentaenoic acid (EPA) and/or docosahexaenoic acid (DHA).

3. The method of claim 1, wherein the administration of the effective amount of the composition comprising phospholipids having bound eicosapentaenoic acid (EPA) and/or docosahexaenoic acid (DHA) improves clearance of α-synuclein aggregation in the subject.

4. The method of any one of claims 1 to 3, wherein the composition comprising phospholipids having bound EPA and/or DHA is characterized in comprising from 5 to 80 g/100 g, from 5 to 60 g/100 g, from 5 to 40 g/100 g, from 8 to 40 g EPA and/or DHA or from 10 to 40 g/100 g EPA and/or DHA as assayed by gas chromatography fatty acid methylester (GC-FAME) analysis.

5. The method of any one of claims 1 to 3, wherein the composition comprising phospholipids having bound EPA and/or DHA is characterized in comprising from 10 to 70%, 10 to 50%, or 10 to 40% EPA and/or DHA w/w where w/w is determined by measuring the total weight of EPA and/or DHA residues in the composition and dividing the total weight of EPA and/or DHA residues by the total weight of the composition.

6. The method of any one of claims 1 to 5, wherein the composition comprising phospholipids having bound EPA and/or DHA is characterized in having a ratio of EPA to DHA of from 1:1 to 5:1 and preferably from 1:1 to 3:1.

7. The method of any one of claims 1 to 5, wherein the composition comprising phospholipids having bound EPA and/or DHA is characterized in having a ratio of DHA to EPA of from 1:1 to 5:1 and preferably from 1:1 to 3:1.

8. The method of any one of claims 1 to 5, wherein the composition comprising phospholipids having bound EPA and/or DHA is further characterized in having a total phospholipid content of from 10 to 90% w/w, 10 to 80% w/w, 20 to 80% w/w, or 20 to 60% w/w, where w/w is determined by measuring the total weight of phospholipids in the composition by 31P-NMR and dividing the total weight of the phospholipid by the total weight of the composition.

9. The method of any one of claims 1 to 8, wherein the composition comprising phospholipids having bound EPA and/or DHA is further characterized in having a choline content of from 2 to 20% w/w, 2 to 15% w/w, or 2 to 10% w/w, where w/w is determined by measuring the total weight of choline in the composition by 31P-NMR and dividing the total weight of the phospholipid by the total weight of the composition.

10. The method of any one of claims 1 to 9, wherein the composition comprising phospholipids having bound EPA and/or DHA is further characterized in comprising astaxanthin esters in an amount of from 50 to 1000 mg/kg, 50 to 500 mg/kg, or 50 to 300 mg/kg, or 100 to 300 mg/kg.

11. The method of any one of claims 1 to 10, wherein the composition comprising phospholipids having bound EPA and/or DHA is further characterized in having a TMAO content, expressed as mgN/100 g, of from 1 to 30, 1 to 20, or 1 to 10 mgN/100 g.

12. The method of any one of claims 1 to 11, wherein the composition comprising phospholipids having bound EPA and/or DHA is further characterized in a peroxide value of less than 10, 5, or 2.

13. The method of any one of claims 1 to 12, wherein the composition comprising phospholipids having bound EPA and/or DHA is further characterized in having an acid value of less than 50, 40, 30 or 20.

14. The method of any one of claims 1 to 13, wherein the composition comprising phospholipids having bound EPA and/or DHA is further characterized in having a Na+ content of less than 1.000%, 0.500%, 0.300% or 0.200% w/w of the composition, where w/w is determined by measuring the total weight of Na+ in the composition and dividing the total weight of Na+ by the total weight of the composition.

15. The method of any one of claims 1 to 14, wherein the composition comprising phospholipids having bound EPA and/or DHA is further characterized in having a phosphatidylcholine (PC) content of from 20 to 70%, 20 to 60%, or 20 to 50% w/w, where w/w is determined by measuring the total weight of PC molecules with two fatty acids attached in the composition and dividing the total weight of PC molecules with two fatty acids attached by the total weight of the composition.

16. The method of any one of claims 1 to 14, wherein the composition comprising phospholipids having bound EPA and/or DHA is further characterized in having a lysophosphatidylcholine (LPC) content of from 10 to 70%, 10 to 60%, or 10 to 50% w/w, where w/w is determined by measuring the total weight of LPC molecules in the composition and dividing the total weight of LPC molecules with one fatty acid attached by the total weight of the composition.

17. The method of any one of claims 1 to 16, wherein the composition comprising phospholipids having bound EPA and/or DHA is prepared from a natural source.

18. The method of claim 17, wherein the natural source is selected from the group consisting of krill, fish or fish by-products, Calanus, shrimp, mollusks, cephalopods, and algae.

19. The method of claim 18, wherein the natural source is krill.

20. The method of claim 18, wherein the fish or fish by-products are derived from herring, salmon, anchovy or mackerel.

21. The method of any one of claims 1 to 20, wherein the composition comprising phospholipids having bound EPA and/or DHA is an oil.

22. The method of any one of claims 1 to 21, wherein the composition comprising phospholipids having bound EPA and/or DHA is encapsulated.

23. The method of claim 22, wherein the composition comprising phospholipids having bound EPA and/or DHA is encapsulated in a gel capsule.

24. The method of any one of claims 1 to 23, wherein the composition comprising phospholipids having bound EPA and/or DHA comprises an antioxidant and/or flavoring agent that does not naturally occur in the source of the phospholipid composition.

25. The method of any one of claims 1 to 24, wherein the composition comprising phospholipids having bound EPA and/or DHA is provided as a medical food.

26. The method of any one of claims 1 to 25, wherein the subject in need thereof has or is at risk of a disease or condition selected from the group consisting of age-related neurodegeneration or disorders, Alzheimer's disease, Parkinson's disease, decline in executive function, age-related cognitive decline, a cognitive disease, disorder or impairment, and senescence related to age-related pathologies selected from the group of atherosclerosis, cardiovascular dysfunction (including cardiovascular problems caused by certain genotoxic chemotherapies), tumor progression, loss of hematopoietic and skeletal muscle stem cell functions, non-alcoholic fatty liver disease, pulmonary fibrosis, osteoarthritis and osteoporosis.

27. Composition comprising phospholipids having bound EPA and/or DHA for use in ameliorating an aging hallmark selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication or for use in improving dopaminergic neuron survival.

28. Use of claim 27, wherein administration of the composition is sufficient to ameliorate at least one of the aging hallmarks.

29. Use of any one of claims 27 to 28, wherein the composition comprising phospholipids having bound EPA and/or DHA is characterized in comprising from 5 to 80 g/100 g, from 5 to 60 g/100 g, from 5 to 40 g/100 g, or from 10 to 40 g/100 g EPA and/or DHA as assayed by gas chromatography fatty acid methylester (GC-FAME) analysis.

30. Use of any one of claims 27 to 28, wherein the composition comprising phospholipids having bound EPA and/or DHA is characterized in comprising from 10 to 70%, 10 to 50%, or 10 to 40% EPA and/or DHA w/w where w/w is determined by measuring the total weight of EPA and/or DHA residues in the composition and dividing the total weight of EPA and/or DHA residues by the total weight of the composition.

31. Use of any one of claims 27 to 30, wherein the composition comprising phospholipids having bound EPA and/or DHA is characterized in having a ratio of EPA to DHA of from 1:1 to 5:1 and preferably from 1:1 to 3:1.

32. Use of any one of claims 27 to 30, wherein the composition comprising phospholipids having bound EPA and/or DHA is characterized in having a ratio of DHA to EPA of from 1:1 to 5:1 and preferably from 1:1 to 3:1.

33. Use of any one of claims 27 to 32, wherein the composition comprising phospholipids having bound EPA and/or DHA is further characterized in having a total phospholipid content of from 10 to 90% w/w, 10 to 80% w/w, 20 to 80% w/w, or 20 to 60% w/w, where w/w is determined by measuring the total weight of phospholipids in the composition by 31P-NMR and dividing the total weight of the phospholipid by the total weight of the composition.

34. Use of any one of claims 27 to 33, wherein the composition comprising phospholipids having bound EPA and/or DHA is further characterized in having a choline content of from 2 to 20% w/w, 2 to 15% w/w, or 2 to 10% w/w, where w/w is determined by measuring the total weight of choline in the composition by 31P-NMR and dividing the total weight of the phospholipid by the total weight of the composition.

35. Use of any one of claims 27 to 34, wherein the composition comprising phospholipids having bound EPA and/or DHA is further characterized in comprising astaxanthin esters in an amount of from 50 to 1000 mg/kg, 50 to 500 mg/kg, or 50 to 300 mg/kg, or 100 to 300 mg/kg.

36. Use of any one of claims 27 to 35, wherein the composition comprising phospholipids having bound EPA and/or DHA is further characterized in having a TMAO content, expressed as mgN/100 g, of from 1 to 30, 1 to 20, or 1 to 10 mgN/100 g.

37. Use of any one of claims 27 to 36, wherein the composition comprising phospholipids having bound EPA and/or DHA is further characterized in a peroxide value of less than 10, 5, or 2.

38. Use of any one of claims 27 to 37, wherein the composition comprising phospholipids having bound EPA and/or DHA is further characterized in having an acid value of less than 50, 40, 30 or 20.

39. Use of any one of claims 27 to 38, wherein the composition comprising phospholipids having bound EPA and/or DHA is further characterized in having a Na+ content of less than 1.000%, 0.500%, 0.300% or 0.200% w/w of the composition, where w/w is determined by measuring the total weight of Na+ in the composition and dividing the total weight of Na+ by the total weight of the composition.

40. Use of any one of claims 27 to 39, wherein the composition comprising phospholipids having bound EPA and/or DHA is further characterized in having a phosphatidylcholine (PC) content of from 20 to 70%, 20 to 60%, or 20 to 50% w/w, where w/w is determined by measuring the total weight of PC molecules with two fatty acids attached in the composition and dividing the total weight of PC molecules with two fatty acids attached by the total weight of the composition.

41. Use of any one of claims 27 to 39, wherein the composition comprising phospholipids having bound EPA and/or DHA is further characterized in having a lysophosphatidylcholine (LPC) content of from 10 to 70%, 10 to 60%, or 10 to 50% w/w, where w/w is determined by measuring the total weight of LPC molecules with one fatty acid attached in the composition and dividing the total weight of LPC molecules with one fatty acid attached by the total weight of the composition.

42. Use of any one of claims 27 to 41, wherein the composition comprising phospholipids having bound EPA and/or DHA is prepared from a natural source.

43. Use of claim 42, wherein the natural source is selected from the group consisting of krill, fish or fish by-products, Calanus, shrimp, mollusks, cephalopods, and algae.

44. Use of claim 43, wherein the natural source is krill.

45. Use of claim 43, wherein the fish or fish by-products are derived from herring, salmon, anchovy or mackerel.

46. Use of any one of claims 27 to 45, wherein the composition comprising phospholipids having bound EPA and/or DHA is an oil.

47. Use of any one of claims 27 to 46, wherein the composition comprising phospholipids having bound EPA and/or DHA is encapsulated.

48. Use of claim 47, wherein the composition comprising phospholipids having bound EPA and/or DHA is encapsulated in a gel capsule.

49. Use of any one of claims 27 to 48, wherein the composition comprising phospholipids having bound EPA and/or DHA comprises an antioxidant and/or flavoring agent that does not naturally occur in the source of the phospholipid composition.

50. Use of any one of claims 27 to 49, wherein the composition comprising phospholipids having bound EPA and/or DHA is provided as a medical food.

51. Use of any one of claims 27 to 50, wherein the composition is administered to a subject that has or is at risk of a disease or condition selected from the group consisting of age-related neurodegeneration or disorders, Alzheimer's disease, Parkinson's disease, decline in executive function, age-related cognitive decline, a cognitive disease, disorder or impairment, and senescence related to age-related pathologies selected from the group of atherosclerosis, cardiovascular dysfunction (including cardiovascular problems caused by certain genotoxic chemotherapies), tumor progression, loss of hematopoietic and skeletal muscle stem cell functions, non-alcoholic fatty liver disease, pulmonary fibrosis, osteoarthritis and osteoporosis.
